(19) United States
(12) Patent Application Publication
(10) Pub. No.: US 2019/0218323 A1
(43) Pub. Date: Jul. 18, 2019

(54) BRANCHED POLYMERS

(71) Applicant: The University of Liverpool, Liverpool-Merseyside (GB)

(72) Inventors: Stephanie Edwards, Liverpool-Merseyside (GB); Steve Rannard, Liverpool-Merseyside (GB); Andrew Owen, Liverpool-Merseyside (GB)

(73) Assignee: The University of Liverpool, Liverpool-Merseyside (GB)

(21) Appl. No.: 16/324,285

(22) PCT Filed: Aug. 8, 2017
(86) PCT No.: PCT/GB2017/052334

§ 371 Date: Feb. 8, 2019

(30) Foreign Application Priority Data
Aug. 8, 2016 (GB) 1613584.0

(51) Int. Cl. C08F 220/28; A61K 47/32; A61K 9/107; C08F 2/38

(52) U.S. Cl. CPC C08F 220/28; A61K 47/32; A61K 9/1075; C08F 2222/1013; C08F 2438/03; C08F 2220/286; C08F 2/38

(57) ABSTRACT

The present invention relates to a branched amphiphilic polymer, suitable for stabilizing an emulsion, comprising a plurality of polymer chains comprising hydrophobic chain ends; a plurality of polymer chains comprising functional chain ends capable of associating to a biological substrate; and a plurality of branching units. The present invention also relates to pharmaceutical compositions containing said branched amphiphilic polymers, their methods of use, and methods for their preparation.

### FIELD OF THE INVENTION

The present invention relates to particular novel branched amphiphilic polymers, compositions containing them, and their methods of use. In addition, the present invention relates to therapeutic methods for the treatment of diseases and to the use of such novel branched amphiphilic polymers in the manufacture of medicaments for use in the treatment and prevention of said diseases.

### BACKGROUND OF THE INVENTION

Branched polymers are polymer molecules of a finite size, which are branched. Branched polymers differ from cross-linked polymer networks which tend towards an infinite size having interconnected molecules and which are generally not soluble but often swellable. In some instances, branched polymers have advantageous properties when compared to analogous linear polymers. For instance, solutions of branched polymers are normally less viscous than solutions of analogous linear polymers. Moreover, higher molecular weight branched polymers can typically be solubilised more easily than corresponding linear polymers. In addition, branched polymers tend to have more end groups than a linear polymer and therefore generally exhibit strong surface-modification properties. Thus, branched polymers are useful components of many, compositions utilised in a variety of fields.

Polymer systems that are capable of forming associations to biological materials and substrates, such as mucous or mucous membranes, offer potential for developing improved delivery methods for biologically active agents or other components. For example, such polymer systems may act by retaining a suitable dosage form at the site of action. Furthermore, such polymer systems may also offer potential for improving systemic delivery and exposure of biologically active agent compounds by promoting diffusion and/or absorption of such compounds across biological surfaces. Retention of suitable dosage forms at such sites in order to achieve these benefits can be challenging and difficulties are often confounded when biologically active agents exhibit challenging physico-chemical properties, for example high lipophilicity and/or poor aqueous solubility. Such agents often require non-conventional and complex drug delivery formulation techniques to provide suitably stable and effective dosage forms. Improvements in residence time at a specific location in the body can yield enhanced delivery and absorption benefits but may also allow the potential for localised and triggered release at these sites of action and/or absorption.

Surprisingly, applicants have found that the particular branched amphiphilic polymers of the invention, which can comprise large numbers of functional moieties capable of forming strong associations to biological substrates, are particularly suitable for preparing highly stable emulsions. Such emulsions can contain biologically active agents and are accordingly useful in pharmaceutical and drug delivery applications. Furthermore, particular emulsions stabilized by the branched amphiphilic polymers of the present invention are also able to selectively breakdown to release their contents upon contact and association with a biological substrate, such as mucous or a mucous membrane. Surprisingly, such demulsification and release is, inter alia, influenced by the size of the emulsion droplets, which can be varied and controlled to provide different release profiles, e.g. a particular release rate or dual release with immediate and sustained or delayed release components.

### SUMMARY OF THE INVENTION

The present invention relates to a branched amphiphilic polymer, suitable for stabilizing an emulsion, comprising a plurality of polymer chains comprising hydrophobic chain ends, a plurality of polymer chains comprising functional chain ends capable of associating to a biological substrate and a plurality of branching units.

In particular embodiments, the functional chain ends are capable of forming a strong association to mucous or a mucous membrane. The polymer chains can be made from vinyl monomers and the hydrophobic chain ends of the polymer chains can be alkyl chains of 5 carbon atoms or more. Conveniently, the functional chain ends of the polymer chains comprise one or more thiol groups.

The invention also relates to processes for the manufacture of said branched amphiphilic polymers and to compositions containing them. In particular, the invention relates to pharmaceutical compositions (such as emulsion compositions) comprising the branched amphiphilic polymer, an effective amount of a biologically active agent or a pharmaceutically acceptable salt thereof, and at least one pharmaceutically acceptable carrier, diluent or excipient. In particular embodiments, the composition is a highly stable oil-in water emulsion formulation that is capable of breaking down to release its contents upon contact and association with a biological substrate, such as mucous or a mucous membrane.

Also in accordance with the present invention there are provided methods of using said branched amphiphilic polymers and compositions containing them in the treatment of diseases.

### BRIEF DESCRIPTION OF DRAWINGS

FIG. 1 shows a schematic representation of a particular branched amphiphilic polymer of the invention, comprising primary linear polymer chains formed from hydrophilic vinyl monomers of poly(ethylene glycol) methyl ether methacrylate, which are branched by the use of ethylene glycol dimethacrylate branching units. Some of the chains have thiol functional chain ends (initiator: Go-BiB) and others have hydrophobic chain ends (initiator: dodecyl bromo isobutyrate, DBiB).

FIG. 2 illustrates some common ways in which conventional oil-in-water emulsions can breakdown.

FIG. 3 is a schematic representation of how a particular branched amphiphilic polymer of the invention may act to stabilize an emulsion at the oil/water interface.

FIG. 4 shows the particle size distributions of emulsions stabilized by a particular branched amphiphilic polymer of the invention (DBiB_{0.25}/SHG_{0(0.75)}(pOEGMA₅₀-co-EGDMA_{0.8}, i.e. thiol content is 75% of chain ends based on molar percent of initiator used during synthesis) after 5 days and 2 weeks post-preparation. The presence of a large number of thiol groups in the branched amphiphilic polymer did not adversely affect the stability of the sample (as assessed by laser diffraction using a Malvern Mastersizer).

FIG. 5 shows that emulsions stabilized by a particular branched amphiphilic polymer of the invention (DBiB_{0.25}/SHG_{0(0.75)}(pOEGMA₅₀-co-EGDMA_{0.8}) are stable (as assessed by optical microscopy) when encapsulated with a hydrophobic drug mimic (both Oil red O and Oil blue O).

FIG. 6. shows that glass slides pre-coated with a layer of mucous when dipped into a concentrated emulsion containing Oil red O stabilized by a particular branched amphiphilic polymer of the invention (DBiB_{(0.25)}/SHG_{0(0.75)}(pOEGMA₅₀-co-EGDMA_{0.8}) exhibit mucoadhesion and triggering of emulsion breakdown over time (FIG. 6 image on left taken immediately after application of the emulsion and image on right taken after 10 minutes). In contrast, when branched polymers only containing hydrophobic chain ends and no functional chain ends (i.e. DBiB) were assessed, no mucoadhesion was observed.

FIG. 7 shows that two separately prepared emulsions with the same thiol content but different coloured dyed oil phases exhibit mucoadhesion and triggered release resulting in mixing of the two coloured dyes. FIG. 7, from left to right shows images taken at 0 mins, 2 mins and 5 mins.

FIG. 8 shows that emulsions exhibit mucoadhesion and triggered release as a result of the emulsion droplets rupturing (optical microscopy images of mucous at 5×, 10× magnification). FIG. 8, from left to right images taken at 0 mins and 5 mins.

FIG. 9 shows that emulsions exhibit mucoadhesion and triggered release as a result of the emulsion droplets rupturing. Optical images were taken at different magnifications to those shown in FIG. 8 in order to provide a broad visual assessment of the emulsion droplets rupturing (optical microscopy images mucous at 10×, 20× magnification) FIG. 9 from left to right shows images taken at 5 mins and 10 mins.

FIG. 10 shows the Z-average diameter (d·nm) of nanoemulsion samples at various ratios of solvent:oil in the dispersed phase stabilized with a thiol containing branched amphiphilic polymer of the invention (DBiB_{0.25}/SHG_{0(0.75)(}pOEGMA₅₀-co-EGDMA_{0.8}).

FIG. 11 shows the particle size distributions (Z-average and polydispersity) of nanoemulsions stabilized by a particular branched amphiphilic polymer of the invention (DBiB_{0.25}/SHG_{0(0.75)}(pOEGMA₅₀-co-EGDMA_{0.8}). The data shows that the presence of the thiol group in the branched amphiphilic polymer did not adversely affect the stability of the nanoemulsion sample.

FIG. 12 compares samples of nanoemulsions stabilized with branched polymers containing hydrophobic chain ends (DBiB 100%) against nanoemulsions stabilized with branched amphiphilic polymers of the current invention (DBiB (0.25)/SHG₀ (0.75), i.e. thiol content is 75% of chain ends based on molar percent of initiator added, composition also contained Oil red O at 0.1 wt % w.r.t. to castor oil). Samples containing only hydrophobic chain ends (i.e. unfunctionalised) do not show any adhesion and the emulsion was easily moved to the sides of the vial on light agitation. In contrast, nanoemulsions containing the functionalised moieties (thiol groups) are highly mucoadhesive.

FIGS. 13a and b show ¹H NMR spectra for Xan₁G_{0(0.75)}/DBiB_{(0.25)}(pOEGMA₅₀-co-EGDMA_{0.8}) and SHG_{0 (0.75)}/DBiB_{(0.25)}(pOEGMA₅₀-co-EGDMA_{0.8}).

FIG. 14 shows the infrared spectra of a first generation dendron used to show the functional group stretches associated with the thiol group.

FIGS. 15a and b show the IR spectra for SHG_{0(0.75)}/DBiB_{(0.25)}(pOEGMA₅₀-co-EGDMA_{0.8}) mixed with excess L-Cysteine and L-Cysteine alone.

FIGS. 16 and 17 show particle size distributions for blank emulsions, emulsions loaded with Amphotericin B, and emulsions loaded with Cyclosporin A, in respect of non-mucoadhesive stabilized macroemulsions (FIG. 16) and mucoadhesive stabilized macroemulsions (FIG. 17).

FIGS. 18 and 19 show particle size distributions for mucoadhesive and non-mucoadhesive stabilized nanoemulsions, loaded with Amphotericin B (FIG. 18) and Cyclosporin A (FIG. 19).

FIG. 20 shows an Amphotericin B fungus kill study including the efficacy of Amphotericin C-loaded emulsions.

FIGS. 21 and 22 show the results of cytotoxicity experiments using nanoemulsions prepared as described herein.

FIG. 23 shows phalloidin staining images of cells treated with Cyclosporin A-loaded emulsions.

### DETAILED DESCRIPTION OF THE INVENTION

### Definitions

The term “association to a biological substrate” refers to association to a material, which is biological in nature, wherein the polymer or composition containing the polymer is held together with the biological material for an extended period of time by interfacial forces. It is to be understood that the term “association” refers to an interaction between the branched amphiphilic polymer of the invention or a composition containing it with the surface of a biological material. The association includes for example adsorption, adhesion, covalent bonding, hydrogen bonding, ionic bonding, electrostatic attraction, Van-der-Waals interaction and polar interactions. The biological substrate can be any biomaterial such as for example mucous, a mucous membrane, the extracellular matrix of a biological cell, bacterial biofilms, mucin layers and keratin (such as present in hair and skin). Conveniently, the association is to mucous or a mucous membrane, the phenomenon typically referred to as mucoadhesion.

Branched Amphiphilic Polymers of the Present Invention

Branched amphiphilic polymers provided by the present invention include those described generally above, and are further illustrated by all classes, subclasses and species of each of these compounds disclosed herein.

Conveniently, the branched amphiphilic polymer is non-gelled, non-crosslinked and processable. It can be contrasted with polymer structures which are insoluble or crosslinked and/or exhibit high viscosity, such as extensively crosslinked insoluble polymer networks, high molecular weight linear polymers, or microgels.

The branched amphiphilic polymer may for example be an addition polymer. The branched amphiphilic polymer may for example be a polymer made from unsaturated, e.g. vinyl or allyl, monomers, such as for example acrylate or methacrylate monomers.

Branched vinyl polymers may be prepared by known methods, from monofunctional vinyl monomers and difunctional vinyl monomers (branching agents). They can be made by, but are not limited to being made by, living polymerisation, controlled polymerisation, step-growth polymerisation or conventional chain-growth polymerisation techniques such as free radical polymerisation. Several types of living and controlled polymerization are known in the art and suitable for use in the present invention. A preferred type of controlled free radical polymerisation is Atom Transfer Radical Polymerisation (ATRP); however other techniques such as Reversible Addition-Fragmentation chain-Transfer (RAFT) and Nitroxide Mediated Polymerisation (NMP) or conventional free-radical polymerisation controlled by the deliberate addition of chain-transfer agents are also suitable syntheses.

The skilled person is aware of techniques to provide branched polymers. For example, suitable procedures are described in N. O'Brien, A. McKee, D. C. Sherrington, A. T. Slark and A. Titterton, Polymer 2000, 41, 6027-6031; T. He, D. J. Adams, M. F. Butler, C. T. Yeoh, A. I. Cooper and S. P. Rannard, Angew. Chem. Int. Ed. 2007, 46, 9243-9247; V. Bütün, I. Bannister, N.C. Billingham, D. C. Sherrington and S. P. Armes, Macromolecules 2005, 38, 4977-4982; I. Bannister, N.C. Billingham, S. P. Armes, S. P. Rannard and P. Findlay, Macromolecules 2006, 39, 7483-7492; and R. A. Slater, T. O McDonald, D. J. Adams, E. R. Draper, J. V. M. Weaver and S. P. Rannard, Soft Matter 2012, 8, 9816-9827.

The polymerization of each vinyl polymer chain starts at an initiator. Copolymerization with difunctional vinyl monomers leads to branching between the chains. In order to control branching and prevent gelation there should be less than one effective brancher (difunctional vinyl monomer) per chain. Under certain conditions, this can be achieved by using a molar ratio of brancher to initiator of less than one: this assumes that the monomer (i.e. the monofunctional vinyl monomer) and the brancher (i.e. the difunctional vinyl monomer) have the same reactivity, that there is no or very limited intramolecular reaction, that the two functionalities of the brancher have the same or similar reactivity, and that reactivity remains the same or substantially unaffected even after part-reaction. Of course, the systems and conditions may be different, but the skilled person understands how to control the reaction and determine without undue experimentation how a non-gelled structure may be achieved. For example, under dilute conditions some branchers form intramolecular cycles which limit the number of branchers that branch between chains even if the molar ratio of brancher to initiator (i.e. polymer chain) is higher than 1:1 in the reaction.

Various initiators and other reagents can be used in the polymerisation process. Mixed initiators can be used to provide different chain end compositions. For example, in ATRP, convenient and effective initiators to introduce hydrophobic chain ends include alkyl halides (e.g. alkyl bromides). In conventional free radical polymerisation, effective initiators include azo compounds. Initiators used to incorporate functional chain ends, such as thiol groups, include but are not limited to xanthate and poly-xanthate initiators, such as for example Xan₁-G₀-BiB, Xan₂-Xan₄-G₂-BiB and Xan₈-G₃-BiB:

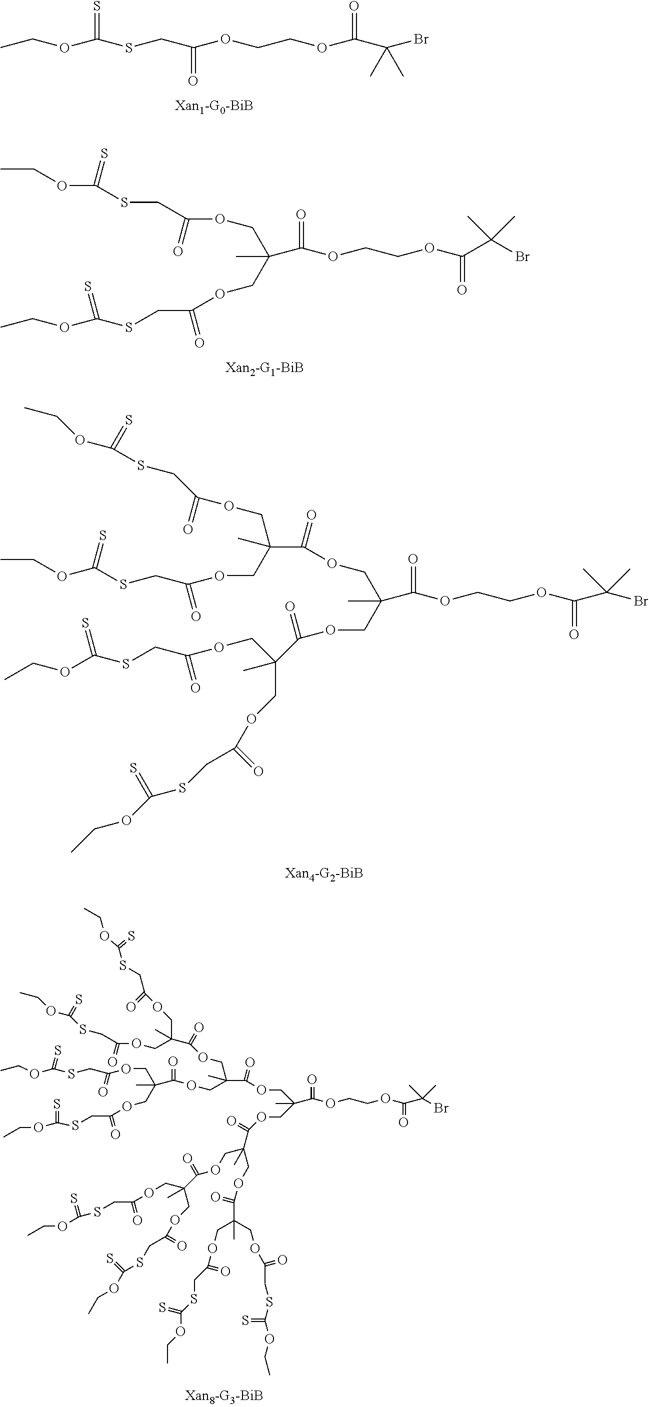 [SMILES: CCOC(=S)SCC(=O)OCCOC(=O)C(C)(C)Br ; CCOC(=S)SCC(=O)OCC(C)(COC(=O)CSC(=S)OCC)C(=O)OCCOC(=O)C(C)(C)Br ; CCOC(=S)SCC(=O)OCC(C)(COC(=O)CSC(=S)OCC)C(=O)OCC(C)(COC(=O)C(C)(COC(=O)CSC(=S)OCC)COC(=O)CSC(=S)OCC)C(=O)OCCOC(=O)C(C)(C)Br ; CCOC(=S)SCC(=O)OCC(C)(COC(=O)CSC(=S)OCC)C(=O)OCC(C)(COC(=O)C(C)(COC(=O)CSC(=S)OCC)COC(=O)CSC(=S)OCC)C(=O)OCC(C)(COC(=O)C(C)(COC(=O)C(C)(COC(=O)CSC(=S)OCC)COC(=O)CSC(=S)OCC)COC(=O)C(C)(COC(=O)CSC(=S)OCC)COC(=O)CSC(=S)OCC)C(=O)OCCOC(=O)C(C)(C)Br]

Other suitable types of branched polymers include branched polyesters. These may be prepared by for example ring opening polymerization of monofunctional lactone monomers and difunctional lactone monomers (branching agents). Ring opening polymerization methods and materials are known in the art, for example from Nguyen et al., Polym Chem 2014, 5, 2997-3008.

One sub-set of suitable branched polymers include those comprising ether or polyether moieties, e.g. those comprising polyethylene glycol (PEG) or polyethylene oxide (PEO), e.g. those made from vinyl monomers comprising ether groups. We have found these to be convenient to prepare and to exhibit good properties, for example when used as emulsifiers in oil-in-water emulsions. Without wishing to be bound by theory, it seems that, whilst the alkyl chains act as anchors in the oil particles, the ether moieties facilitate stabilisation in water. Suitable monomers for use in a method of preparing branched polymers having PEG groups include PEG-acrylate or other vinyl versions of PEG. One particular example of a suitable monomer for use in a method of preparing branched polymers having PEG groups is oligo(ethylene glycol) methacrylate (OEGMA), also known as PEG-methacrylate.

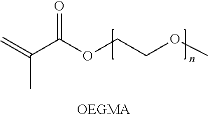

Use of this monomer allows the incorporation of multiple ether moieties. This monomer already contains a number of ethylene oxide moieties. For example, a PEG-methacrylate with Mn=5000 g/mol has an n number of approximately 115.

In one particular embodiment described herein, polymerisation and branching is carried out simultaneously by mixing mono-functional and bifunctional monomers in a single feed. However, the introduction of branches can be achieved after polymerisation of the primary vinyl chains. Indeed, this monomer can be polymerised via its vinyl moiety such that, before connection of the primary vinyl polymer chains via branches, it may contain for example 5 to 500 OEGMA units. Conveniently, the degree of polymerisation (DPₙ) of the primary chains of the branched amphiphilic polymer of the invention is between 50 and 100 monomer units.

Other suitable monofunctional monomers include, but are not limited to, for example N-butyl methacrylate, N-butyl acrylate, N-butyl methacrylamide 2-hydroxypropyl methacrylate, 2-hydroxypropyl acrylate, N,N-diethyl amino ethyl methacrylate, N,N-diethyl amino ethyl acrylate, glycerol methacrylate, glycerol acrylate and 2-methacryloyloxyethyl phosphorylcholine. Mixtures of different monomers may be used so as to form a copolymer.

Suitable types of difunctional monomer (i.e. brancher) include for example those which comprise two or more polymerisable functional groups e.g. acrylate, acrylamide or methacrylate monomers.

One example of a suitable brancher is ethylene glycol dimethacrylate (EGDMA). This is convenient and effective.

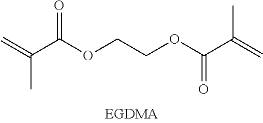

Other suitable examples of branchers include, but are not limited to, oligoethylene glycol dimethacrylate, polyethylene glycol dimethacrylate, bisphenol A dimethacrylate, polydimethylsiloxane dimethacrylate, divinyl benzene, oligoethylene glycol diacrylate, polyethylene glycol diacrylate, bisphenol A diacrylate and polydimethylsiloxane diacrylate.

Hydrophobic Chain Ends

The branched amphiphilic polymer of the present invention can be understood to comprise a number of primary polymer chains held together with branches between the chains (preferably one branch or fewer per chain). In a particular embodiment, some of the chains ends are hydrophobic alkyl moieties. Such hydrophobic alkyl chain ends may be incorporated into the branched polymer via a suitable initiator or chain transfer agent, e.g. via bromide initiators (such as bromo isobutyrates) or mercaptan chain transfer agents. An initiator (or chain transfer agent) may include an alkyl chain of 5 carbon atoms or more. Conveniently, the initiator is selected from dodecyl bromo isobutyrate (DBiB), hexyl bromo isobutyrate (HBiB) and ethyl bromo isobutyrate (EBiB).

For use as an emulsifier in an oil-in-water emulsion, this is a convenient and effective way of imparting the required hydrophobic character so as to stabilise or “anchor into” the oil droplets. Furthermore, it is flexible: it enables the alkyl chain ends of the resultant polymer to be varied easily, simply by varying the initiator, and thereby provides an important means of tailoring the composition. In a particular embodiment, these hydrophobic “anchors” do not need to be present on each polymer chain. Surprisingly, when used as an emulsifier in oil-in-water emulsions, effective emulsification and effective stability can be achieved when 90% or fewer, or 75% or fewer, or 50% or fewer, or even 25% or fewer, of the polymer chain ends carry the required alkyl chain. This means that, where the hydrophobic moieties are incorporated via initiators, only some of the initiators are required to carry these hydrophobic moieties.

Functional Chain Ends

The branched amphiphilic polymers of the current invention also comprise functional moieties. Indeed, the branched amphiphilic polymers comprise a plurality of polymer chains comprising functional chain ends capable of associating to a biological substrate.

Such functional moiety can be incorporated into the branched amphiphilic polymer via an initiator or chain transfer agents, e.g. via bromide initiators (such as bromo isobutyrates) or mercaptan chain transfer agents. Thus, an initiator (or chain transfer agent) may include a functional moiety capable of associating to a biological substrate. Conveniently, the initiator employed to provide the functional moiety is selected from Xan₁-G₀-BiB, Xan₂-G₁-BiB, Xan₄-G₂-BiB and Xan₈-G₃-BiB. Conveniently, the initiator employed to provide the functional moiety is Xan₁-G₀-BiB.

Other mucoadhesive functionalities include charged polymers, such as poly(acrylic acid). Polymers that have an overall negative net charge at pH values exceeeding the pKa of the polymer, with for example the presence of carboxyl and sulphate functional agroups can be suitable (Andrew, G. et al., 2008, Eu. Jr. Phrm. Biophrm., 71, 505-518). Other mucoadhesive functionalities include polyacrylates (Khutoryanskiy, V., 2010, Macromolecular Bioscience, 11(6), 748-7) or polyethylene glycol modified poly(lactic-co-glycolic)acid polymers (Cu, Y., Saltzman, W. M, 2008, molecular pharmaceutics, 6(1),173-181).

For use as emulsifiers in an oil-in-water emulsion, branched amphiphilic polymers that comprise both hydrophobic alkyl chains and a functional moiety capable of associating to a biological substrate provide a convenient and effective way of imparting the required hydrophobic character so as to stabilise or “anchor” the oil droplets but also provide the ability to target and associate with a biological substrate. Such polymers can be synthesised with two initiators at different ratios to give the two different required chain end compositions.

The branched amphiphilic polymers of the present invention are capable of associating with a biological substrate. Such functionality is achieved by polymer chain ends comprising a functional moiety capable of associating to a biological substrate. Such association provides the potential to optimize localized drug delivery of biological agents, by retaining a suitable dosage form at the site of action (e.g. within any of the sites within the gastro-intestinal tract, including the mouth, stomach, intestine and colon), the bladder, the surface of the eye, the respiratory system (including the nasal cavity and the mucosal surfaces of the lungs), the skin, hair or parts of the reproductive organs (such as the vagina) or improving systemic delivery by promoting absorption across various biological surfaces. Retention of suitable dosage forms at such sites can be challenging, for example in the eye, where many drugs are quickly eliminated via the lacrimal gland (Urtti, A., 2006, Adv. Drug. Deliv. Rev. 58, 1131-1135) or where formulations are unable to adsorb to the highly hydrophobic surface of the eye. Improving retention of suitable dosage forms at such sites can improve delivery and absorption of biologically active agents but also allows the potential for targeted and triggered release at the sites of action. Conveniently, the suitable dosage form in this embodiment is an emulsion formulation.

The branched amphiphilic polymers could also be used as therapeutic agents in their own right, for example to coat and protect damaged tissues (gastric ulcers or lesions of the oral mucosa) or to act as lubricating agents (in the oral cavity, eye and vagina).

In a particular embodiment, the biological substrate to which the functional moiety capable of associating is mucous or a mucous membrane. Mucous membranes (mucosae) are the moist surfaces lining the walls of various body cavities such as the gastrointestinal and respiratory tracts. They consist of a connective tissue layer (the lamina propria) above which is an epithelial layer, the surface of which is made moist usually by the presence of a mucous layer. The epithelia may be either single layered (e.g. the stomach, small and large intestine and bronchi) or multilayered/stratified (e.g. in the oesophagus, vagina and cornea). The former contain goblet cells which secrete mucous directly onto the epithelial surfaces, the latter contain, or are adjacent to tissues containing, specialized glands such as salivary glands that secrete mucous onto the epithelial surface. Mucous is present as either a gel layer adherent to the mucosal surface or as a luminal soluble or suspended form. As mentioned above, mucous can present a barrier for local and systemic drug delivery.

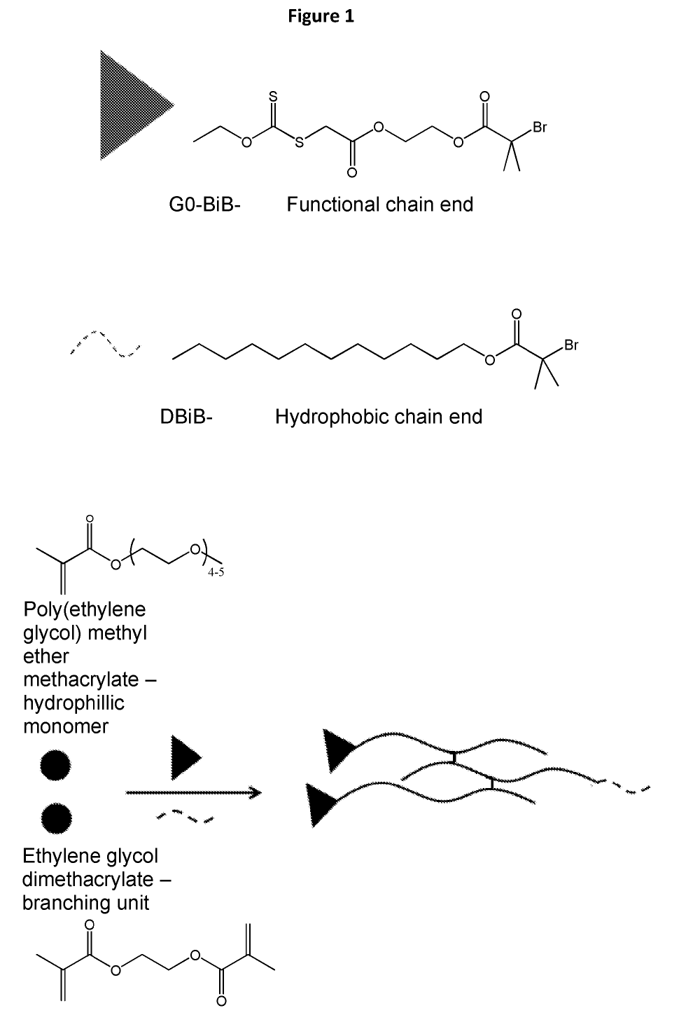

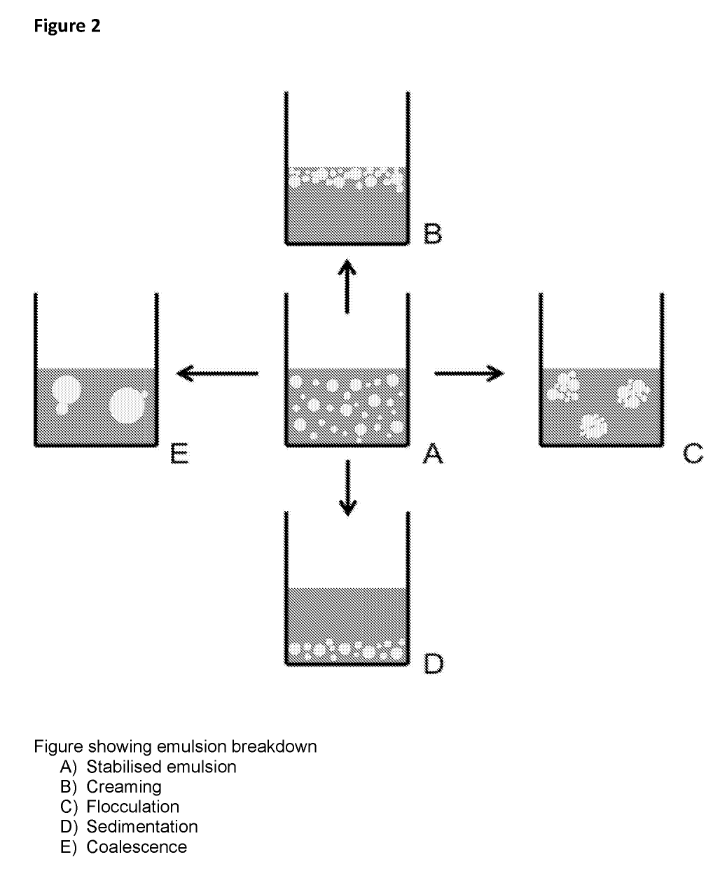

In one embodiment, the functional moiety capable of associating to a biological substrate comprises thiol groups. Surprisingly, the inventors have found that by incorporating such groups into at least one or more ends of the polymer chains of the branched amphiphilic polymer, the branched amphiphilic polymer is particularly useful in providing a means to target and associate with a biological substrate, such as for example mucous or a mucous membrane. Conveniently, the thiol groups are incorporated into the polymer chains as xanthate functional groups. FIG. 1 provides details of a suitable branched polymer of the invention comprising the hydrophilic monomer poly(ethylene glycol) methyl ether methacrylate used to prepare the polymer chains, G₀-BiB initiators used to provide functional chain ends comprising a xanthate functional group, DBiB used as initiator to provide hydrophobic chain ends and ethylene glycol dimethacrylate as branching unit. Surprisingly, inventors have found that when branched amphiphilic polymers of the invention are deprotected to remove the xanthate and generate thiol functional groups and are employed as emulsifiers for oil-in-water emulsions encapsulating hydrophobic materials, the emulsion droplets associate with a biological substrate, such as for example mucous or a mucous membrane. Furthermore, droplets can rupture with time to provide a triggered release of their contents (e.g. biologically active agent). This targeted release provides a number of potential benefits for localized and systemic drug delivery applications.

Surprisingly, when used as an emulsifier in oil-in-water emulsions, effective emulsification and high stability can be achieved even when one or more of the polymer's chains comprise a functional moiety capable of associating to a biological substrate. In one particular embodiment, 10-90% of the polymer chain ends (based on the molar percent of initiator used during synthesis) carry the functional moiety. In a further embodiment, at least 50%, 60% or 70% of the polymer chain ends carry functional chain ends. In yet a further embodiment, 70-80% (conveniently 75%) of the polymer chain ends carry functional chain ends. Conveniently, the functional moieties are thiol groups.

Pharmaceutical Compositions

In another aspect, there is provided a pharmaceutical composition comprising a branched amphiphilic polymer, an effective amount of a biologically active agent or a pharmaceutically acceptable salt thereof, and at least one pharmaceutically acceptable carrier, diluent, or excipient.

In one aspect, there is provided a pharmaceutical composition comprising an effective amount of a branched amphiphilic polymer, a biologically active agent or a pharmaceutically acceptable salt thereof, and at least one pharmaceutically acceptable carrier, diluent, or excipient for use as a medicament.

As used herein, the phrase “effective amount” means an amount of a biologically active agent or composition containing a biologically active agent which is sufficient enough to significantly and positively modify the symptoms and/or conditions to be treated (e.g. provide a positive clinical response). The effective amount of the biologically active agent for use in a pharmaceutical composition will vary with the intended therapeutic or prophylactic purpose, the particular condition being addressed, the severity of the condition, the duration of the treatment, the nature of concurrent therapy, the particular biologically active agent(s) being employed, the particular pharmaceutically-acceptable excipient(s)/carrier(s) utilized, and like factors within the knowledge and expertise of the attending physician. As used herein, the term “pharmaceutically acceptable” refers to those compounds (for example biologically active agent compounds described herein), materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

It is to be appreciated that references to “treat”, “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms. Conveniently, particular pharmaceutical compositions of the invention can be used for prophylaxis. In one particular embodiment, pharmaceutical compositions of the invention are used for prophylaxis to prevent the spread or transmission of infectious disease. For example, such compositions can be topical dosage forms for vaginal application.

Conveniently, the biologically active agent is a lipophilic drug with poor aqueous solubility. Non-limiting examples of suitable biologically active agents include for example the antiretroviral drugs Lopinavir (LPV) and Efavirenz (EFV), and the antibiotics Rifampicin and Erythromycin.

Compositions of the invention may be in a form suitable for oral use, for topical use (for example as creams, ointments, dermal and transdermal patches, gels, or aqueous or oily solutions or suspensions), for administration by inhalation (for example as a finely divided powder or a liquid aerosol), for administration by insufflation (for example as a finely divided powder), for parenteral administration (for example as a sterile aqueous or oily solution for intravenous, subcutaneous, or intramuscular dosing) or as a suppository for rectal dosing or pessary for vaginal administration. Conveniently, compositions of the present invention are administered by oral administration.

The amount of biologically active ingredient that is combined with one or more excipients to produce a single dosage form will necessarily vary depending upon the host being treated with the therapeutic or prophylactic composition and the particular route of administration. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990. The size of the dose required for the therapeutic or prophylactic treatment of a particular disease state will also necessarily be varied depending on the host, the route of administration and the severity of the illness. The optimum dosage may be determined by the practitioner who is treating any particular patient.

Emulsion Compositions

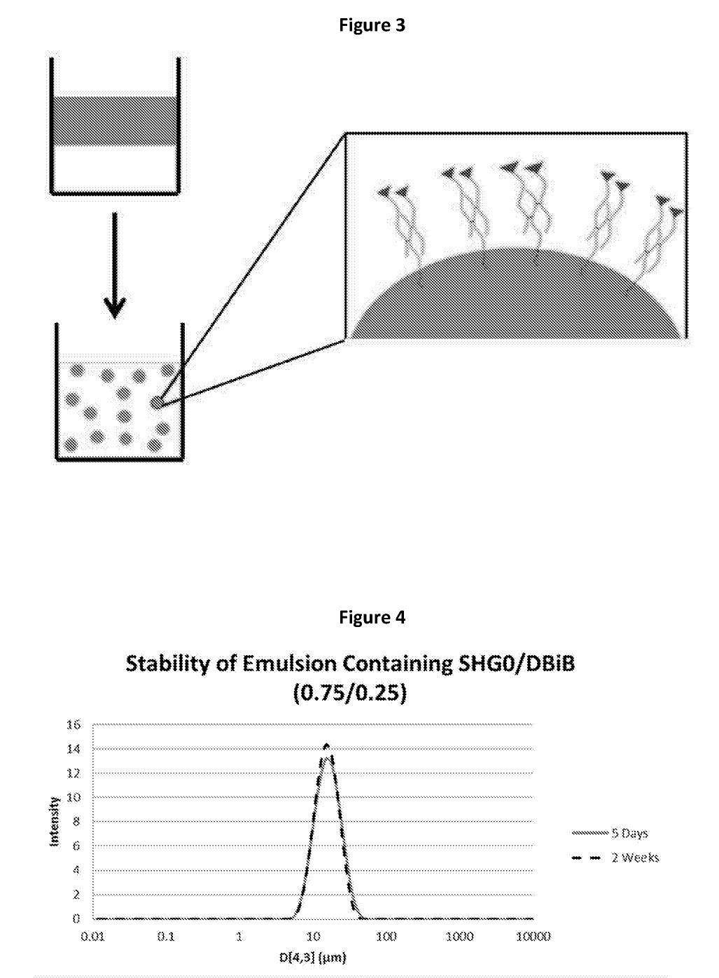

In a particular embodiment, the branched amphiphilic polymer is employed in an emulsion composition and the emulsions are of particular use in the pharmaceutical and drug delivery areas. Emulsions are typically highly unstable mixtures of two immiscible phases, which wish to be separate. A high input of energy is often required in order to prevent separation, which can occur via various different mechanisms. FIG. 3 illustrates some common ways in which emulsions can breakdown. Classical emulsions include mixtures of oil (dispersed phase) and water (continuous phase) and polymeric surfactants can be used to stabilise the two phases. Such polymeric surfactants can reside at the interface between oil and water to give a monodispersed oil phase. Classical emulsions also include emulsions where a hydrophilic phase is dispersed in a hydrophobic phase (water-in-oil) or double emulsions, so called water-in-oil-in-water or the converse oil-in-water-in-oil emulsions. The formation of stable emulsions requires the use of materials which can absorb at the biphasic interface and prevent emulsion breakdown.

Surprisingly, the inventors have found that the branched amphiphilic polymer of the current invention can comprise a low percentage of hydrophobic chain ends and still provide a storage stable dispersed phase even though the remaining chain ends comprise a large number of functional moieties capable of associating to a biological substrate. FIG. 3 is a schematic representation of how a particular branched amphiphilic polymer of the invention may act to stabilize an emulsion at the oil/water interface, where the low percentage of hydrophobic chain ends act as anchors into the oil droplet and serve to stabilize the emulsion.

Conveniently, the present invention provides an oil-in-water emulsion as described above, for use as a medicament. The present invention also provides a corresponding method of medical treatment comprising administration of an effective amount of an oil-in-water emulsion as defined above, to a subject in need thereof. The compositions are particularly effective in oral drug delivery. The inventors have found that the emulsions are surprisingly effective in not only maintaining excellent stability but also in associating to a biological substrate and therefore optimising delivery and treatment.

A further aspect the present invention provides a method of preparing an oil-in-water emulsion comprising mixing an oil phase with an aqueous phase in the presence of a branched amphiphilic polymer, wherein said branched amphiphilic polymer is a non-gelled branched polymer comprising a plurality of polymer chains comprising hydrophobic chain ends, a plurality of polymer chains comprising functional chain ends capable of associating to a biological substrate and a plurality of branching units.

In an initial step of preparing the emulsions, the biologically active agent and/or other component may be dissolved in an oil. For pharmaceutical uses and therapeutic administration the oil must of course be selected from oils that are suitable for, and safe for, those applications. The skilled person is well aware of oils that fulfil this criterion. Advantageously the oil will be a good solvent for the material to be carried. Some suitable oils include castor oil, coconut oil, dodecanoic acid, squalene, peanut oil, sesame oil and soy bean oil. Castor oil is particularly preferred with some biologically active agents. Saturating the oil with the drug will give the maximum possible concentration of the biologically active agent in the final emulsion. The emulsion can easily be diluted if required.

Formulations containing various hydrophobic materials are carried, for example Curcumin, Flourescein and Nile Red can also be prepared.

Optionally a further solvent may be used during the preparation procedure in addition to the oil. The solvent is typically miscible with the oil and would not adversely affect the solubility of the biologically active agent in the mixture. Typically, the further solvent would not be present in the final emulsion and is therefore one which can be removed by evaporation or other methods.

Suitable volatile solvents include for example ethyl acetate, hexane, acetone or THF. Ethyl acetate is one preferred solvent as it is not miscible with water, it is miscible with many oils, has appreciable water-solubility, evaporates easily and quickly, and has low toxicity.

The oil (in which the biologically active agent or other hydrophobic material is dissolved) is mixed with the volatile solvent. The ratio of oil to solvent can be selected to tailor the size of the emulsion droplets. Typically, the higher the solvent to oil ratio, the smaller the droplets in the final emulsion. The amount, by volume, of solvent with respect to oil may for example be 50:50 or greater, e.g. 60:40 or greater, e.g. 70:30 or greater, e.g. 80:20 or greater, e.g. 90:10 or greater, e.g. 95:5 or greater, e.g. 99:1 or greater, e.g. 95:5 to 99.9:0.1, e.g. approximately 99:1.

The emulsion is conveniently formed by mixing the oil phase (which optionally includes the volatile solvent) with an aqueous solution of the branched amphiphilic polymer.

The amount of aqueous phase relative to oil phase, and the concentration of the branched amphiphilic polymer within the aqueous phase, can be chosen to tailor the nature and properties of the emulsion. Sufficient polymer should be used to stabilise the emulsion droplets. The concentration of polymer can affect the size of the droplets. Without wishing to be bound by theory, it is believed that lower amounts of polymer lead to larger droplets due to there not being enough polymer to fully encapsulate the droplets and therefore leading to aggregation. Conversely, there is typically an upper limit of polymer required, such that above that amount no further stabilisation benefit will be observed and leading to free polymer in solution.

In some cases, the preferred concentration of polymer in the aqueous phase (w/v) is selected from approximately 0.1-99.9%, 0.5-99%, 1-90%, 1-50%, 1-20%, 2-10%, 3-7%, or approximately 5%.

In some cases, the amount of oil (plus optional solvent) phase relative to the amount of aqueous phase (v/v) is approximately 90:10 to 10:90, or 75:25 to 25:75, or 60:40 to 40:60, or approximately 50:50.

The oil phase and aqueous phase may be mixed and homogenised using any suitable method or apparatus to result in an oil-in-water emulsion. The emulsion can comprise nanosized droplets and these can be determined by appropriate light scattering or laser diffraction methods.

When present, the volatile solvent may be removed by any suitable method, for example by allowing it to evaporate, and/or by dilution and stirring and/or by passing gas (e.g. inert gas e.g. nitrogen) through the material. Conveniently, the material can be simply left in unsealed containers to allow evaporation (e.g. in a fume cupboard) over a period of 12-48 hours, typically about 24 hours.

Evaporation or removal of the solvent leads to formation of the emulsion in its final form. This has the biologically active agent and/or other hydrophobic component(s) present in the oil phase, which oil phase is stabilised in water due to the interaction of the polymer and the nanodroplets. In one embodiment, the z-average diameter of the emulsion droplets, as determined by dynamic light scattering (DLS), is typically less than 1000 nm. In a different embodiment, the z-average diameter of the emulsion droplets, as determined by dynamic light scattering (DLS), is typically between 1-100 μm. The z-average diameters may be measured by DLS at 25° C.

Particular emulsions of the invention are stable on storage and on dilution.

The oil-in-water emulsion may have particles or droplets of different sizes to those described above, i.e. not necessarily having a z-average diameter of no greater than about 1000 nm or between 1-100 μm. In one particular embodiment, the oil-in-water emulsion has a mixture of particles or droplets with different sizes to provide a controlled and tailored release profile (e.g. a dual release system comprising both immediate release and sustained or delayed release components).

Accordingly, a further aspect the present invention provides an oil-in-water emulsion, comprising an emulsifier, which is a branched amphiphilic polymer as described herein. Other features of such emulsions, and of corresponding compositions, uses and methods, may be as described above.

In a particular embodiment, a pharmaceutical composition may contain two or more separately prepared emulsions compositions, in which each emulsion composition contains a different biologically active agent and/or other hydrophobic component(s). In this particular embodiment, the different biologically active agent and/or other hydrophobic component(s) can be kept separate in stable emulsions until contact with a biological substrate such as mucous or a mucous membrane, at which point the emulsions stabilized by the branched amphiphilic polymers of the present invention can selectively breakdown and release their contents. This can be particularly advantageous where such biologically active agent(s) and/or other hydrophobic component(s) would otherwise be unstable if co-formulated together.

In addition to pharmaceutical uses, the emulsions of the invention may also be useful in other areas where benefit from the stability and/or functional association with biological substrates is advantageous. For example, the emulsions of the invention may also be useful in mouthwash compositions, agrochemicals, veterinary applications, cosmetics and other consumer goods products, such as cleansing creams, ointments, pastes, lotions and shampoos.

### Examples

Further examples of the invention are described hereinbelow, by way of example only, with reference to the accompanying figures.

Preparation of Initiators

### Dodecyl α-bromoisobutyrate (DBiB)

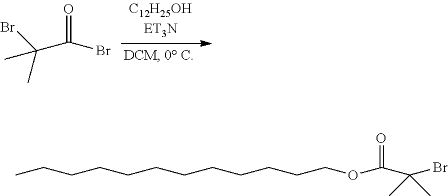

1-dodecanol (9.32 g, 50 mmol, 1.0 eqiv), triethylamine (6.07 g, 60 mmol, 1.2 eqiv) were dissolved in dichloromethane (70 mL). α-bromoisobutyl bromide (13.80 g, 60 mmol, 1.2 eqiv) was added dropwise via a pressure equalising dropping funnel and stirred in an ice bath under nitrogen. After addition reaction vessel was left to warm to room temperature and left to stir for 24 hours. The solution was washed once with NaHCO₃ (50 mL), distilled water (4×50 mL). The organic layer was dried over anhydrous MgSO₄, filtered, and concentrated in vacuo. If ¹H NMR showed the need for additional purification, product was passed through a basic alumina column. Yield: 3.155 g, yellow oil, (19%). ¹H NMR (400 MHz, CDCl₃) δ=0.81 (t, 3H), 1.19 (m, 18H), 1.61 (m, 2H), 1.86 (s, 6H), 4.08 (t, 2H). ¹³C NMR (100 MHz, CDCl₃)=14.1, 22.7, 25.8, 28.4, 29-30, 32.0, 56.0, 66.2, 171.8. Calcd. [M+H]⁺ (C₁₆H₃₂BrO₂) m/z=335.0. Found: ES MS [M+H]⁺ m/z=335.2. Anal. Cald. (C₁₆H₃₁O₂Br)═C, 57.31; H, 9.32. Found=C, 57.33; H, 9.20.

### 2-((Ethoxycarbonothioyl)thio) Acetic Acid

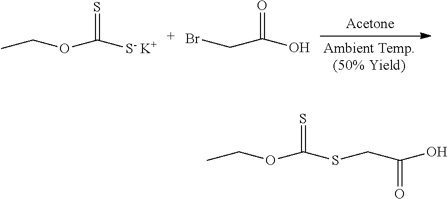

Potassium ethyl xanthogenate (53.06 g, 311 mmol, 1 eqiv.) was stirred acetone (400 mL). A solution of 2-bromoacetic acid (38.35 g, 276 mmol, 1.12 eqiv.) in acetone (100 mL) was added dropwise to the main reaction vessel over 20 minutes and left to stir at ambient temperature for 16 hours. The crude mixture was filtered under vacuum, washed through with acetone and solvent removed. The residual oil was diluted in dichloromethane and washed with brine (150 mL). The organic layer was dried over MgSO₄ and solvent removed to give a white solid. Yield: 16.28 g, white solid, (33%). ¹H NMR (400 MHz, CDCl₃) δ=1.43 (t, 3H), 3.98 (s, 2H), 4.65 (q, 2H), 9.3 (s, br, —OH). ¹³C NMR (100 MHz, CDCl₃) δ=13.70, 37.63, 70.93, 173.86, 212.07. Anal. Cald. (C₅H₈O₃S₂)=C, 33.32; H, 4.47; S, 35.58. Found=C, 32.80; H, 4.40; S, 34.59. Xan₁-G₀-BiB

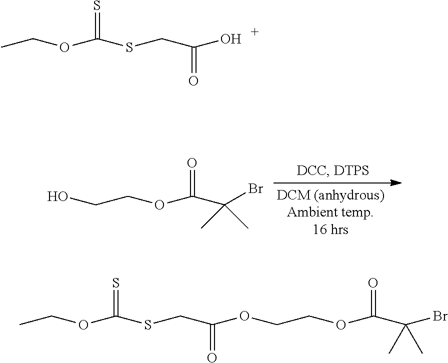

2-((Ethoxycarbanothioyl)thio) acetic acid, XanCOOH (3.85 g, 21.2 mmol, 1 eqiv), 2-hydroxyethyl 2-bromoisobutyrate (4.5 g, 21.2 mmol, 1 eqiv) and 4-(dimethylamino)pyridinium-4-toluene sulphonate (DPTS) (6.86 g, 23.32 mmol, 1.1 eqiv) were dissolved in anhydrous dichloromethane (40 mL) under nitrogen. N,N′-Dicyclohexylcarbodiimide (4.81 g, 23.32 mmol, 1.1 eqiv) was dissolved in anhydrous dichloromethane (10 mL) under nitrogen flow and transferred to main reaction vessel via syringe and the reaction was left to stir at ambient temperature for 16 hours. The resulting crude mixture was filtered, diluted in dichlormethane (100 mL) and washed with distilled water (2×100 mL) and once with brine (100 mL). The organic layer was dried over MgSO₄. After removal of solvents the xanthate initiator was purified by automated liquid chromatography (silica, eluting hexane increasing the polarity to hexane:ethyl acetate 70:30) to give pure product. Yield: 5.08 g, yellow oil, (25%). ¹H NMR (400 MHz, CDCl₃) δ=1.43 (t, 3H), 1.94 (s, 6H), 3.96 (s, 4H), 4.41 (m, 4H), 4.66 (q, 2H). ¹³C NMR (100 MHz, CDCl₃) δ=Calcd. [M+Na]⁺ (C₁₁H₁₇BrO₅S₂Na) m/z=395.28. Found: ES MS [M+Na]⁺ m/z=395. Anal. Cald. (C₁₁H₁₇BrO₅S₂)=C, 35.39; H, 4.59; S, 17.18. Found=C, 36.29; H, 4.79; S, 17.06.

### 2-hydroxyethyl 2-bromoisobutyrate

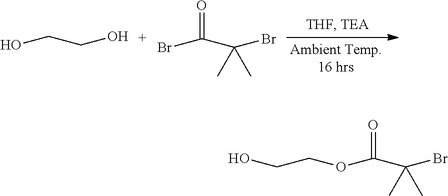

Ethylene glycol (301.35 g, 4855 mmol, 50 eqiv.), triethylamine (20.33 g, 201 mmol, 2 eqiv.) were dissolved in anhydrous tetrahydrafuran (100 mL) and the reaction was stirred in an ice bath. A-bromoisobutyrl bromide (22.32 g, 97.1 mmol, 1 eqiv.) was added dropwise over 30 minutes and the reaction was left stirring under nitrogen atmosphere at ambient temp for 16 hours. Reaction mixture was poured into distilled water (800 mL) and extracted with dichlormethane (6×100 mL), layers washed with 1M HCL (2×300 mL), dried over MgSO₄ and solvent removed. Yield: 18.60 g, yellow oil, (90.80%). ¹H NMR (400 MHz, CDCl₃) δ=1.96 (s, 6H), 3.87 (t, 2H), 4.31 (t, 2H). ¹³C NMR (100 MHz, CDCl₃) δ=30.7, 55.8, 60.7, 63.3, 67.4, 171.8. Calcd. [M+H]⁺ (C₆H₁₂BrO₃) m/z=211.0. Found: CI MS [M+Na]⁺ m/z=211.0 Anal. Cald. (C₆H₁₁BrO₃)=C, 34.14; H, 5.25. Found=C, 34.09; H, 5.24.

Preparation of Branched Amphiphilic Polymers

DBiBₓ/Xan₁-G₀-BiB_{y}(pOEGMA₅₀-co-EGDMA_{0.8})

Where x+y=1 eqiv., and represent the molar ratio of the initiators DBiB:Xan₁-G₀-BiB. This ratio can be varied and ranges have been generated from 0.9:0.1, 0.75:0.25, 0.5:0.5, 0.25:0.75, 0.1:0.9 DBiB:Xan₁-Gₒ-BiB as well as the homopolymers of each.

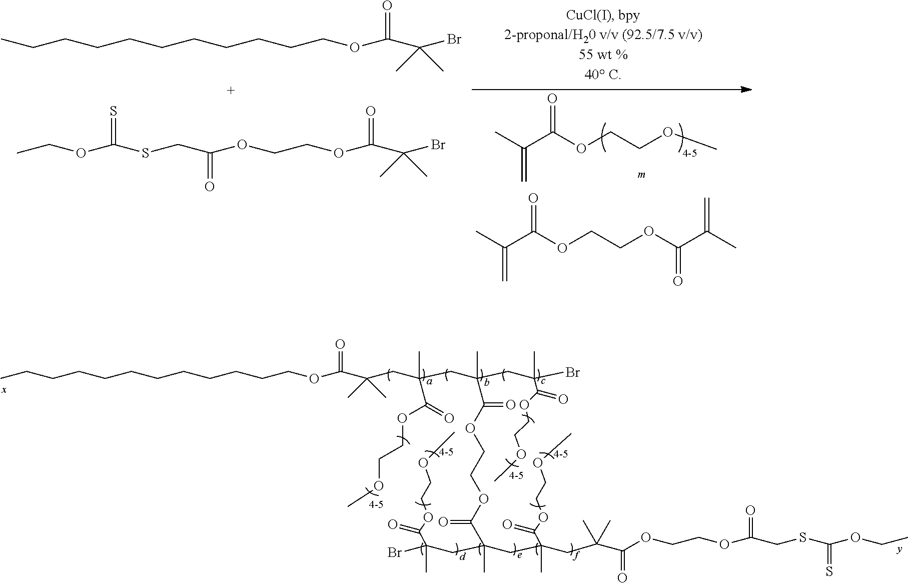

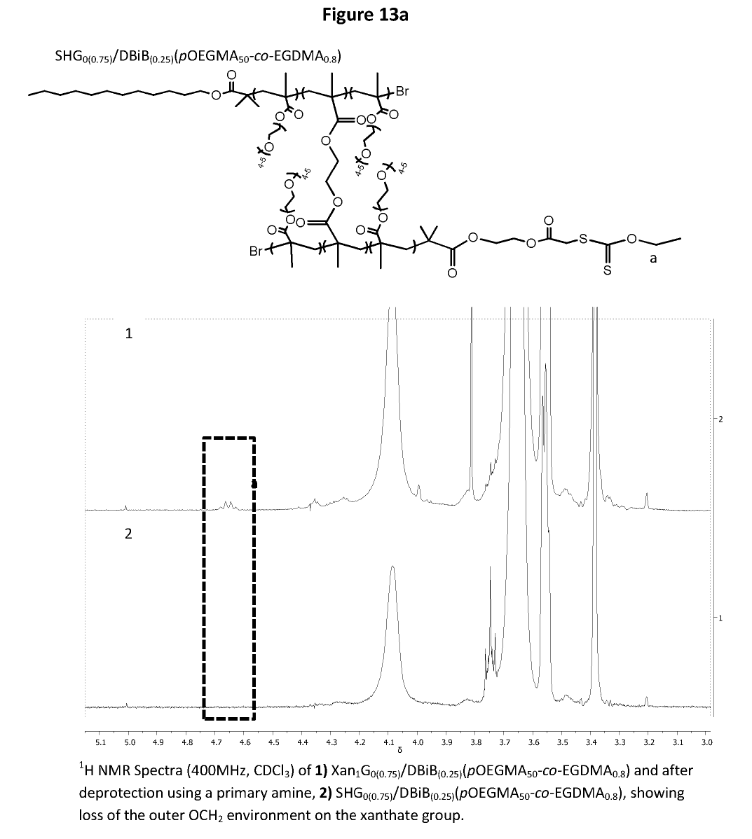

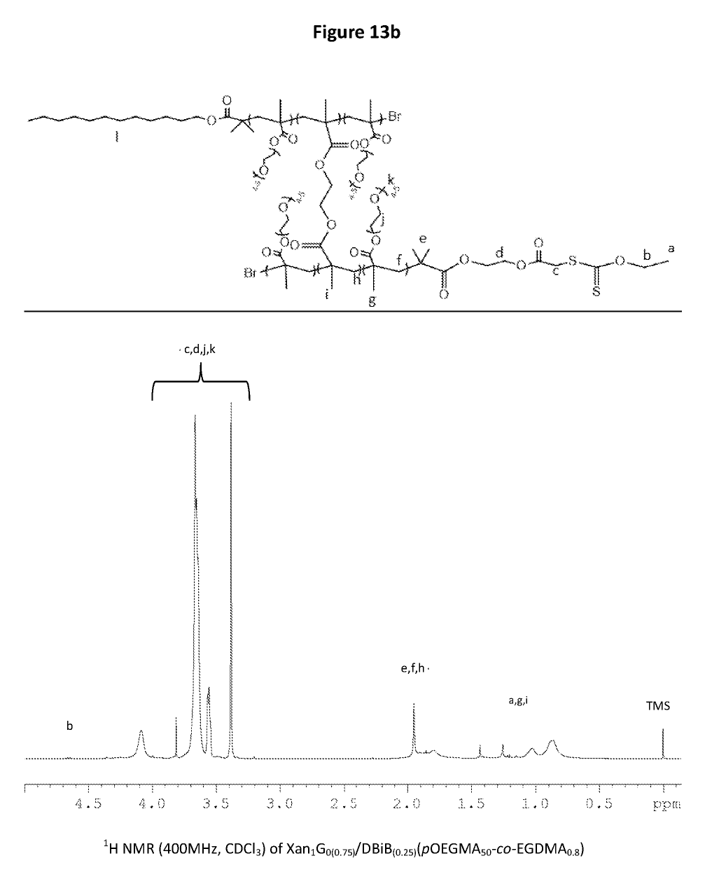

In a typical atom-transfer radical polymerisation reaction, OEGMA (5.00 g, 16 mmol, 50 eq.), EGDMA (0.051 g, 0.32 mmol, 0.8 eq.), 2,2′-bipyridyl (0.100 g, 0.64 mmol, 2 eq.), DBiB (0.0268 g, 0.08 mmol, 0.25 eq.), Xan-G₀-BiB (0.089 g, 0.24 mmol, 0.75 eq.) were added to a RBF equipped with magnetic stirrer and IPA/H₂O (92.5:7.5 v/v, 4.39:0.36 mL, 55 wt %) added as solvent. The vessel was sealed and degassed with dry nitrogen for 5 minutes, CuCl(I) (0.032 g, 0.32 mmol, 1 eq.) was added and the reaction vessel sealed. RBF was immersed in silicon oil bath at 40° C. and left to react until complete conversion, approx. 24 hrs. Anisole was added as internal standard for ¹H NMR conversion of monomer peaks. Polymerisation terminated by exposure to air and dilution in THF. Copper catalyst removed by neutral alumina column, solvent removed in vacuo and crude polymer precipitated twice into cold hexane. Residual solvent removed in vacuo. ¹H NMR Spectra are provided in FIGS. 13a and b.

DBiB(pOEGMA₅₀) or Xan₁-G₀-BiB(pOEGMA₅₀)

In a typical atom-transfer radical polymerisation reaction, OEGMA (5.00 g, 16 mmol, 50 eq.), 2,2′-bipyridyl (0.100 g, 0.64 mmol, 2 eq.), DBiB (0.0268 g, 0.08 mmol, 0.25 eq.) or Xan₁-G₀-BiB (0.089 g, 0.24 mmol, 0.75 eq.) were added to a RBF equipped with magnetic stirrer and IPA/H₂O (92.5:7.5 v/v, 4.39:0.36 mL, 55 wt %) added as solvent. The vessel was sealed and degassed with dry nitrogen for 5 minutes, CuCl(I) (0.032 g, 0.32 mmol, 1 eq.) was added and the reaction vessel sealed. RBF was immersed in silicon oil bath at 40° C. and left to react until complete conversion, approx. 24 hrs. Anisole was added as internal standard for ¹H NMR conversion of monomer peaks. Polymerisation terminated by exposure to air and dilution in THF. Copper catalyst removed by neutral alumina column, solvent removed in vacuo and crude polymer precipitated twice into cold hexane. Residual solvent removed in vacuo.

Deprotection of DBiBₓ/Xan₁-Gₒ-BiB_{y}(pOEGMA₅₀-co-EGDMA_{0.8})

In a typical experimental procedure to remove the xanthate protecting group, DBiBₓ/Xan₁-Gₒ-BiB_{y}(pOEGMA₅₀-co-EGDMA_{0.8}) (0.599 g, 1.66 mmol, 1 eqiv.) was dissolved in tetrahydrafuran (10 mL) and degassed with dry nitrogen for ˜5 minutes. Butyl amine (0.38 mL, 4.15 mmol, 2.5 eqiv.) was added to the reaction vessel and left to stir for 1.5 hrs. Solvent removed and crude product precipitated twice into cold hexane. Residual solvent removed in vacuo.

Preparation of Macroemulsions

Aqueous polymer solutions were prepared at 5 mg/mL of branched amphilic polymer for the water phase of the emulsion. Emulsions were prepared at a 1:1 v:v ratio of oil:water, where the oil phase was dodecane. Emulsions were homogenised via over-head shear homogenisation (IKA T 25 ULTRA-TURRAX) for 2 minutes at 24,000 rpm. Emulsions are left over night before characterisation of droplet using light scattering was carried out (Malvern mastersizer 2000).

FIG. 4 shows the particle size distributions of emulsions stabilized by a particular branched amphiphilic polymer of the invention (DBiB_{0.25}/SHG_{0(0.75)}(pOEGMA₅₀-co-EGDMA_{0.8}, i.e. thiol content is 75% of chain ends based on molar percent of initiator used during synthesis) after 5 days and 2 weeks post-preparation. The presence of a large number of thiol groups in the branched amphiphilic polymer did not adversely affect the stability of the sample (as assessed by laser diffraction using a Malvern Mastersizer).

Examples Using Encapsulated Hydrophobic Drug Mimics

Preparation of Macroemulsions with Encapsulated Hydrophobic Drug Mimic Oils

Aqueous polymer solutions were prepared at 5 mg/mL of branched amphiphilic polymer for the water phase of the emulsion. Emulsions were prepared at a 1:1 v:v ratio of oil:water, where the oil phase was dodecane. Oil red 0 or Oil blue 0 (0.5 wt % w.r.t. dodecane) were used as a hydrophobic drug mimic. Emulsions were homogenised via over-head shear homogenisation (IKA T 25 ULTRA-TURRAX) for 2 minutes at 24,000 rpm. Emulsions are left over night before characterisation of droplet using light scattering was carried out (Malvern mastersizer 2000).

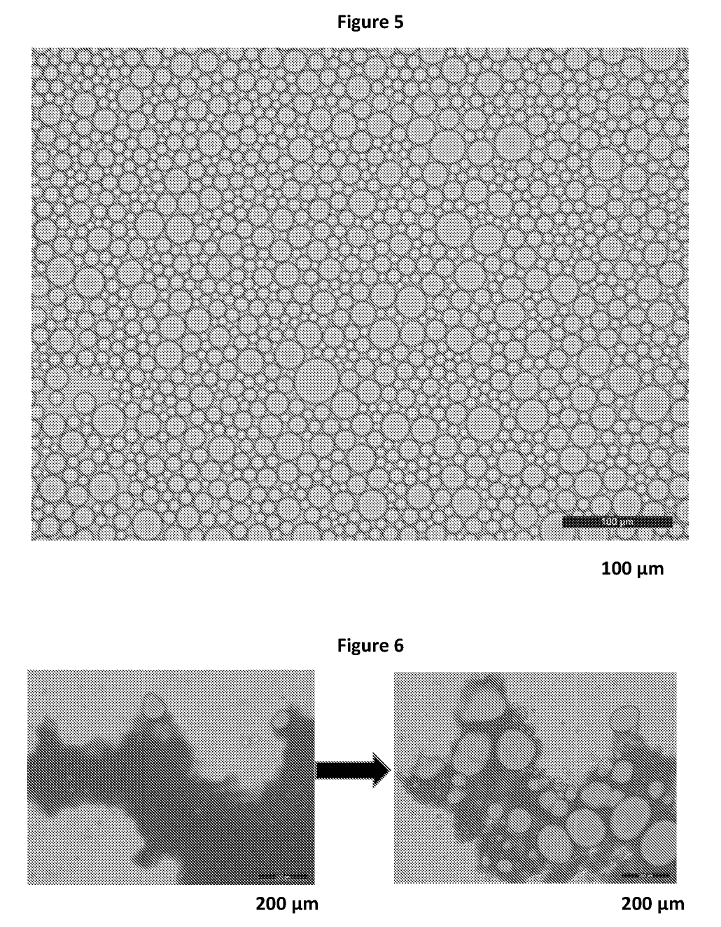

FIG. 5 shows that emulsions stabilized by a particular branched amphiphilic polymer of the invention (DBiB_{0.25}/SHG_{0(0.75})(pOEGMA₅₀-co-EGDMA_{0.8}) are stable (as assessed by optical microscopy) when encapsulated with a hydrophobic drug mimic (both Oil red O and Oil blue O).

Mucoadhesion Assessment

Biosimilar mucous was synthetically prepared to mimic that which is normally secreted in the gastro-intestinal tract. The mucous was prepared with porcine mucin/lipids found in natural mucous, which contains cysteine. The procedure used is described in Boegh. M et al., 2014, European journal of Pharmaceutics and Biopharmaceutics, 87(2), 227-235. The biosimilar mucous was coated onto glass slides or poured into glass vials and used for the emulsion assessments as described below.

FIG. 6. shows that glass slides pre-coated with a layer of mucous when dipped into a concentrated emulsion containing Oil red O stabilized by a particular branched amphiphilic polymer of the invention (DBiB_{(0.25)}/SHG_{0(0.75)}(pOEGMA₅₀-co-EGDMA_{0.8}) exhibit mucoadhesion and triggering of emulsion breakdown over time (FIG. 6 image on left taken immediately after application of the emulsion and image on right taken after 10 minutes). In contrast, when branched polymers only containing hydrophobic chain ends and no functional chain ends (i.e. DBiB) were assessed, no mucoadhesion was observed.

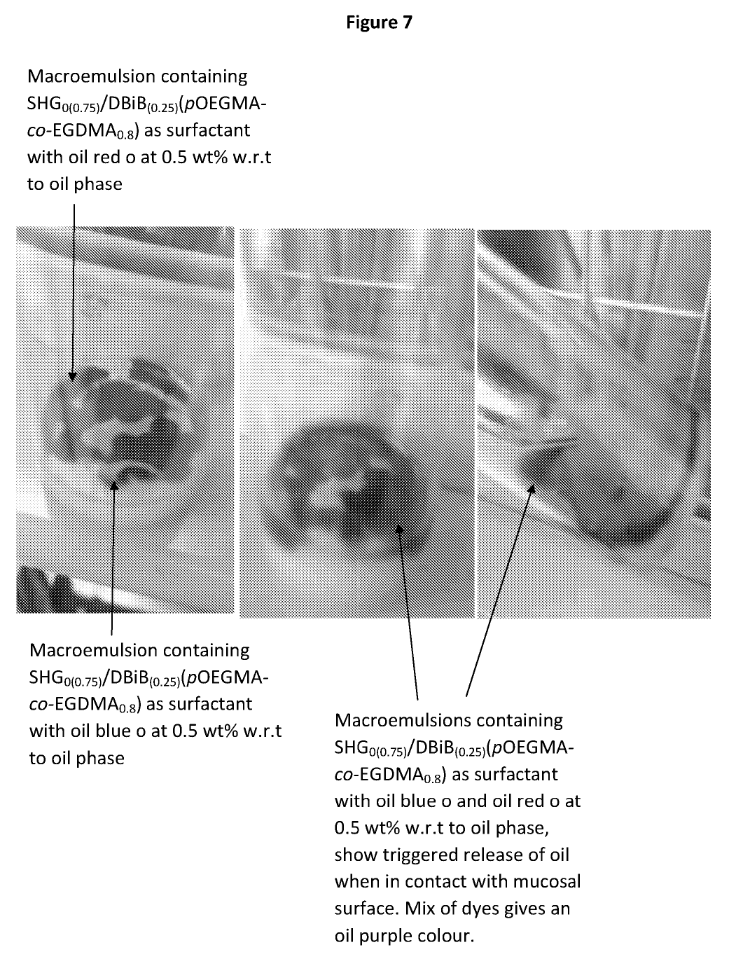

FIG. 7 shows that two separately prepared emulsions with the same thiol content but different coloured dyed oil phases exhibit mucoadhesion and triggered release resulting in mixing of the two coloured dyes. Emulsions used are oil-in-water emulsions at 1:1 ratio of oil:water where the dispersed phase is dodecane and the continuous phase is an aqueous polymer solution. The branched amphiphilic polymer used is DBiB_{(0.25)}/SHG_{0(0.75)}(pOEGMA₅₀-co-EGDMA_{0.8}) at 5 mg/mL w.r.t. to aqueous phase. Oil red O or Oil blue O was incorporated at 0.5 wt. % w.r.t. to oil phase. Emulsions were homogenized via over-head shear homogenisation (IKA T 25 ULTRA-TURRAX) for 2 minutes at 24,000 rpm. 100 μl of each emulsion was applied to the surface of the biosimilar mucous. FIG. 7, from left to right shows images taken at 0 mins, 2 mins and 5 mins.

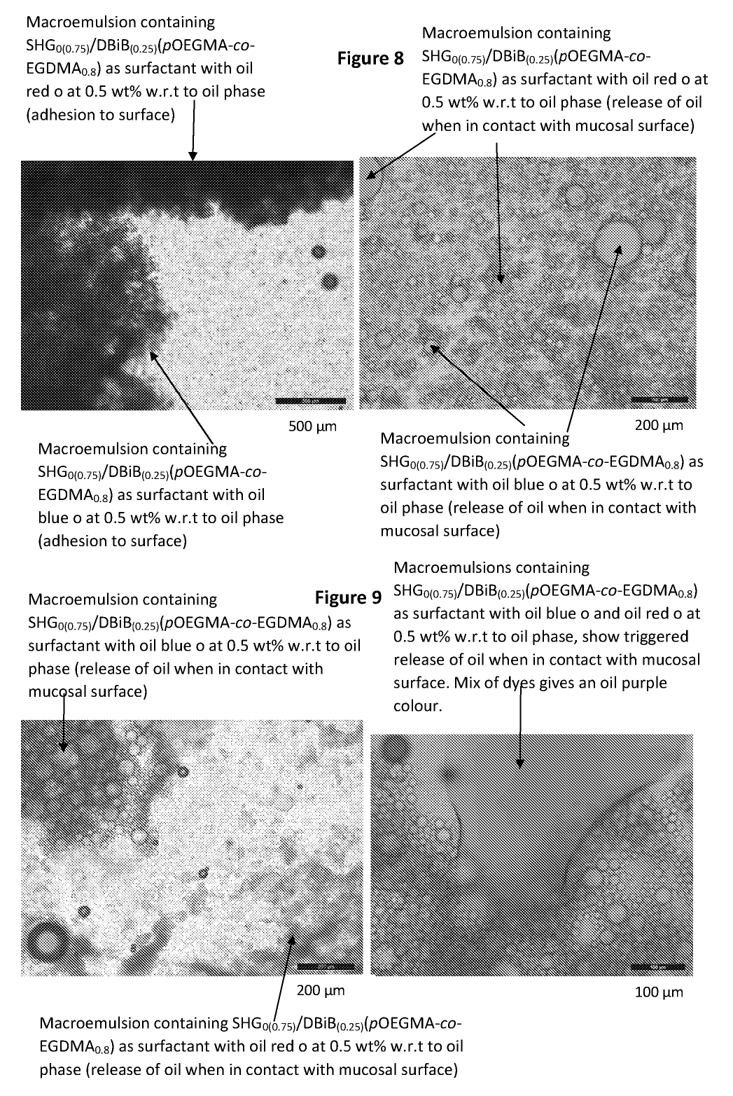

FIG. 8 shows that emulsions exhibit mucoadhesion and triggered release as a result of the emulsion droplets rupturing (optical microscopy images of mucous at 5×, 10× magnification). Emulsions used are oil-in-water emulsions at 1:1 ratio of oil:water where the dispersed phase is dodecane and continuous phase is aqueous polymer solution. The branched amphiphilic polymer used is DBiB_{(0.25)}/SHG_{0(0.75)}(pOEGMA₅₀-co-EGDMA_{0.8}) at 5 mg/mL w.r.t. to aqueous phase. Oil red O or Oil blue O was incorporated at 0.5 wt % w.r.t. to oil phase. Emulsions were homogenized via over-head shear homogenisation (IKA T 25 ULTRA-TURRAX) for 2 minutes at 24,000 rpm. 100 μl of each emulsion was applied to the surface of the biosimilar mucous. FIG. 8, from left to right images taken at 0 mins and 5 mins.

FIG. 9 shows that emulsions exhibit mucoadhesion and triggered release as a result of the emulsion droplets rupturing. Optical images were taken at different magnifications to those shown in FIG. 8 in order to provide a broad visual assessment of the emulsion droplets rupturing (optical microscopy images mucous at 10×, 20× magnification) Emulsions used are oil-in-water emulsions at 1:1 ratio of oil:water where the dispersed phase is dodecane and continuous phase is aqueous polymer solution. The branched amphiphilic polymer used is DBiB_{(0.25)}/SHG_{0 (0.75)}(pOEGMA₅₀-co-EGDMA_{0.8}) at 5 mg/mL w.r.t. to aqueous phase. Oil red 0 or Oil blue 0 was incorporated at 0.5 wt % w.r.t. to oil phase. Emulsions homogenized via over-head shear homogenisation (IKA T 25 ULTRA-TURRAX) for 2 minutes at 24,000 rpm. 100 μl of each emulsion was applied to the surface of the biosimilar mucous. FIG. 9 from left to right shows images taken at 5 and 10 mins.

Nanoemulsion Formulations

Nanoemulsions were formulated using the solvent evaporation technique. The oil phase is a mixture of two miscible oils, one a volatile solvent. As the volatile solvent evaporates, the non-volatile section of the oil droplet shrinks to the nanoscale. The oil phase is composed of ethyl acetate:castor oil in a ratio of 50:50, 60:40, 70:30, 80:20, 90:10 or 99:1. The oil:water ratio was 1:1, with the water phase being aqueous polymer solution at 5 wt %. Emulsions formulated via homogenisation using an over-head shear homogeniser (IKA T 25 ULTRA-TURRAX) for 2 minutes at 24,000 rpm. Emulsions are left overnight until all ethyl acetate is removed and analysis performed using dynamic light scattering (malver, zetasizer nano)

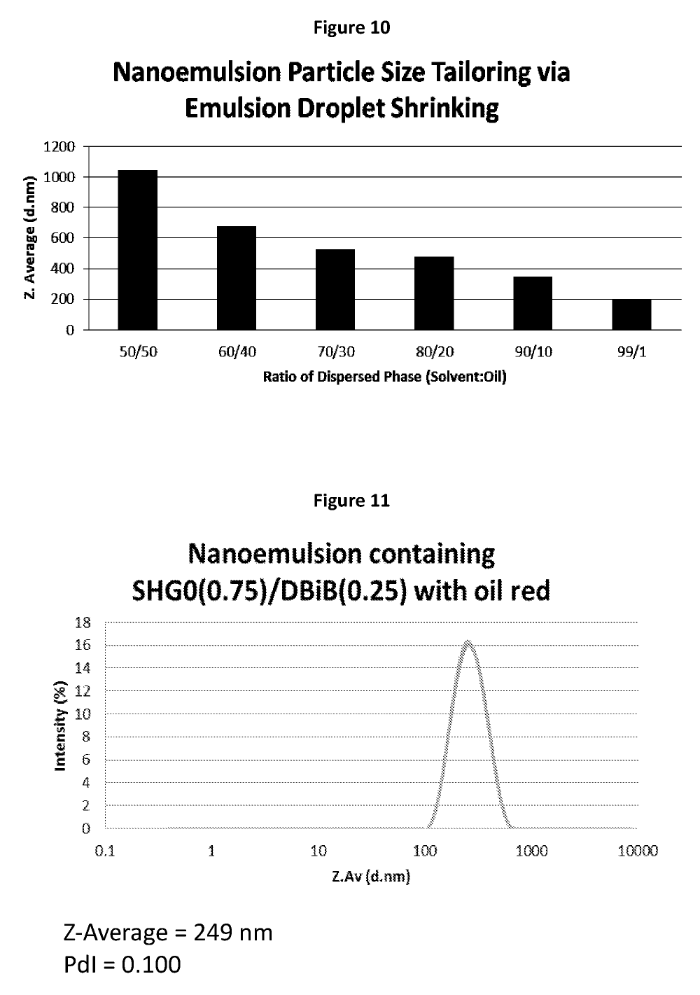

FIG. 10 shows the Z-average diameter (d.nm) of nanoemulsion samples at various ratios of solvent:oil in the dispersed phase stabilized with a thiol containing branched amphiphilic polymer of the invention (DBiB_{0.25}/SHG_{0(0.75)}(pOEGMA₅₀-co-EGDMA_{0.8}).

FIG. 11 shows the particle size distributions (Z-average and polydispersity) of nanoemsulsions stabilized by a particular branched amphiphilic polymer of the invention (DBiB_{0.25}/SHG_{0(0.75)}(pOEGMA₅₀-co-EGDMA_{0.8}). In the final emulsion, the continuous phase is water and the dispersed phase is castor oil stabilized by the branched amphiphilic polymer. In order to prepare the nanoemulsion, the branched amphiphilic polymer was used as an aqueous polymer solution at 5 wt % and the dispersed phase was ethyl acetate/castor oil (99:1 ratio). The ethyl acetate was removed during the process resulting in a size reduction of the emulsion droplets into the nano-range. The data shows that the presence of the thiol group in the branched amphiphilic polymer did not adversely affect the stability of the nanoemulsion sample.

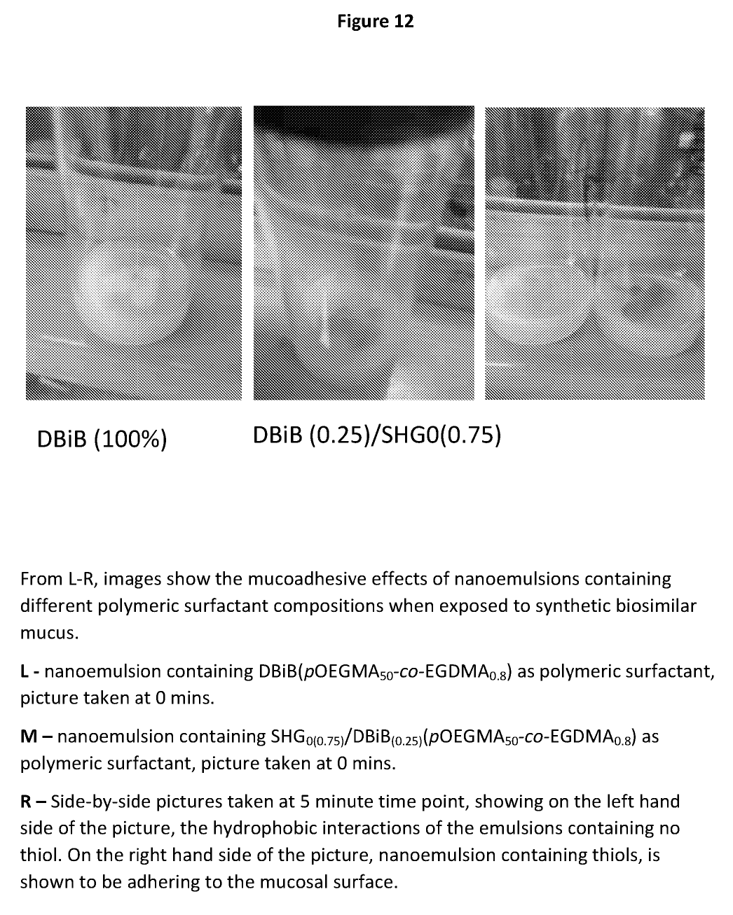

FIG. 12 compares samples of nanoemulsions stabilized with branched polymers containing hydrophobic chain ends (DBiB 100%) against nanoemulsions stabilized with branched amphiphilic polymers of the current invention (DBiB (0.25)/SHG₀ (0.75), i.e. thiol content is 75% of chain ends based on molar percent of initiator added, composition also contained Oil red 0 at 0.1 wt % w.r.t. to castor oil). Samples containing only hydrophobic chain ends (i.e. unfunctionalised) do not show any adhesion and the emulsion was easily moved to the sides of the vial on light agitation. In contrast, nanoemulsions containing the functionalised moieties (thiol groups) were highly mucoadhesive. The methodology employed was a simple visual mucoadhesion experiment, in which two sample vials containing biosimilar mucous (1 mL) were set up. To each vial emulsions containing polymeric surfactants DBiB(pOEGMA₅₀-co-EGDMA-0.8) or DBiB_{0.25}/SHG_{0(0.75)}(pOEGMA₅₀-co-EGDMA_{0.8}) (100 μl) were added respectively. Over 5 minutes the samples were monitored to witness any adhesion to the surface of the synthetic mucous.

Disulphide Bond Formation Between Thiolated Polymer and Mucus

IR experiments were carried out to further show the disulphide bond formation that occurs between the thiolated polymer and the cysteine component of the synthetic biosimilar mucus.

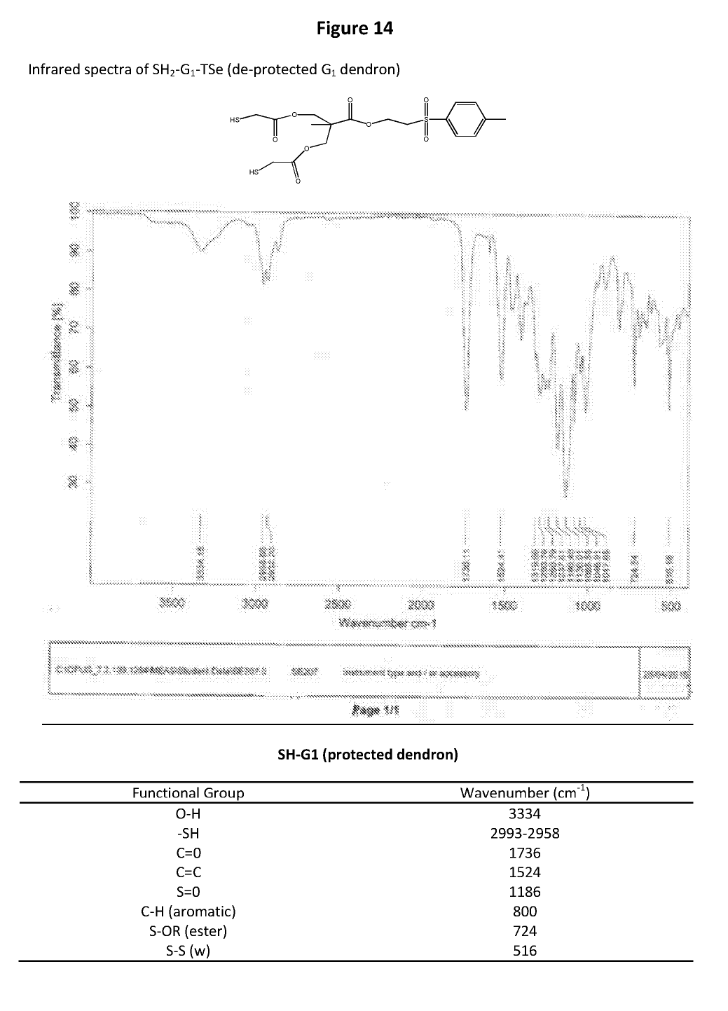

Firstly, Infrared Spectrometry (IR) was used in proof of concept experiments to confirm that free thiols groups could be analysed i.e. IR was used to monitor how xanthate terminated dendrons can be deprotected to expose thiol groups. FIG. 14 shows the infrared spectra of a first generation dendron used to show the functional group stretches associated with the thiol group. The structure (shown below), is the first generation dendron which is protected at the focal point before conversion to an ATRP macroinitiator.

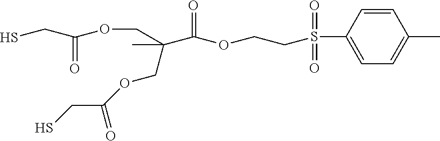 [SMILES: Cc1ccc(S(=O)(=O)CCOC(=O)C(C)(COC(=O)CS)COC(=O)CS)cc1]

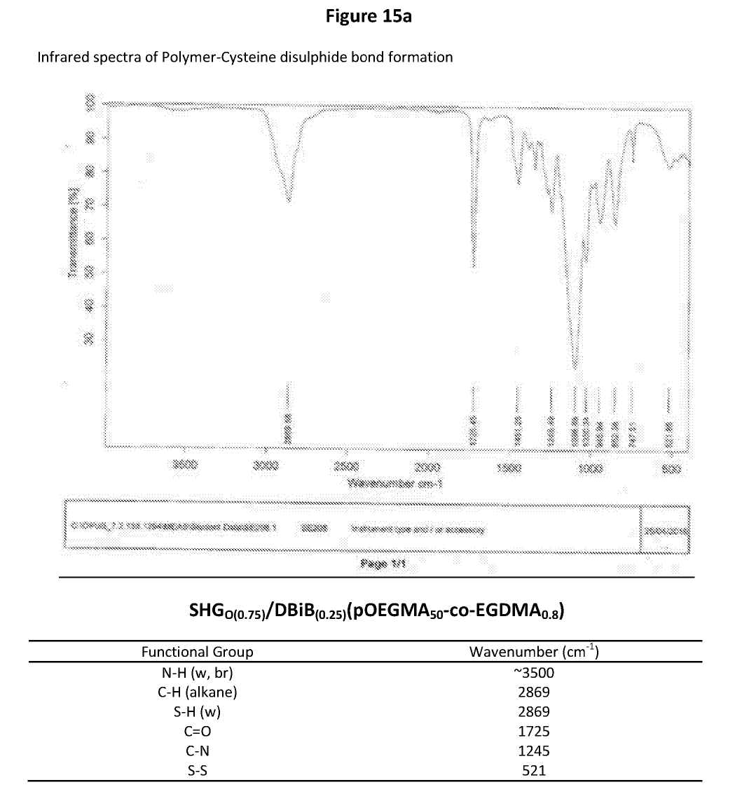

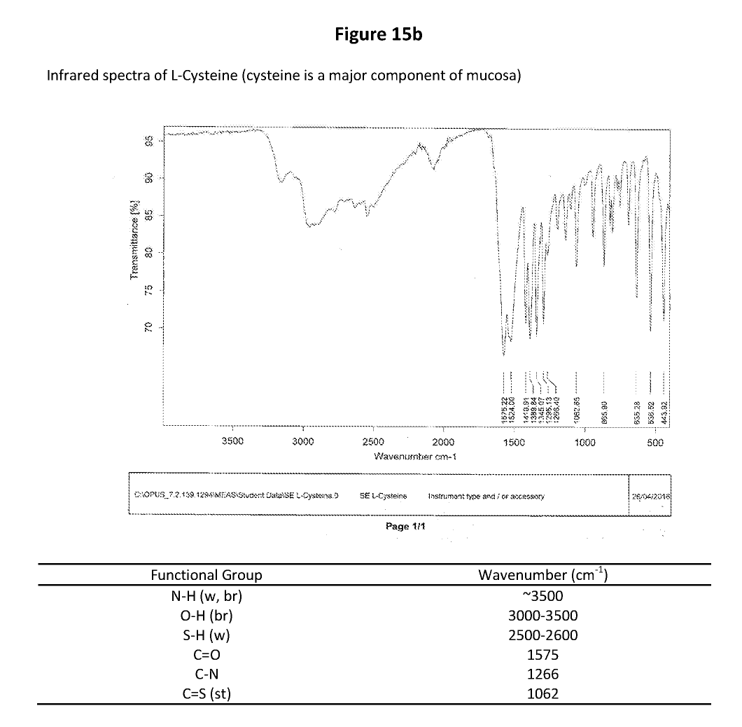

To perform the bond formation study, SHG_{0 (0.75)}/DBiB_{(0.25)}(pOEGMA₅₀-co-EGDMA_{0.8}) (0.41 g, 0.0011 mmol, 1 eqiv.) and excess L-Cysteine (2.06×10⁻⁴ g, 0.0017 mmol, 1.5 eqiv.) were mixed in water for ˜1.5 hrs. Solvent was removed in vacuo. IR spectra are shown in FIGS. 15a and b and confirm the addition of N—H, C—N and S-S stretches. The data demonstrates the disulphide bond formation between the mucoadhesive polymer and cysteine, which is a major component of mucosal surfaces.

Examples Using Encapsulated Drugs

Some of the examples above utilise hydrophobic drug mimics. Further experiments were carried out using drugs including Amphotericin B and Cyclosporin A.

Polymer Synthesis

DBiBₓ/Xan₁-G₀-BiB_{y}(pOEGMA₅₀-co-EGDMA_{0.8}) was prepared and deprotected as described above (“Preparation of branched amphiphilic polymers”)

Preparation of Macroemulsions

Macroemulsions were prepared as described above, except that the oil phase was changed from dodecane to squalene due to this being more biologically favourable.

Macroemulsions with Encapsulated Drugs for Ocular Drug Delivery

Aqueous polymer solutions were prepared at 5 mg/mL of branched amphiphilic polymer for the water phase of the emulsion. Emulsions were prepared at a 1:1 v:v ratio of oil:water, where the oil phase was squalene. Amphotericin B and Cyclosporin A were loaded in the oil phase at the clinical topical dose concentration, 0.15% w/v and 0.05% w/v respectively. Emulsions were homogenised via over-head shear homogenisation (IKA T 25 ULTRA-TURRAX) for 2 minutes at 24,000 rpm. Emulsions were left over night before characterisation of droplet size using light scattering was carried out (Malvern mastersizer 2000).

Results

For the following experiments a comparison of a non-mucoadhesive emulsion stabilized by branched polymer DBiB(pOEGMA₅₀-co-EGDMA_{0.8}) against a mucoadhesive emulsion stabilized by DBiB_{25/}SHG₀BiB₇₅(pOEGMA₅₀-co-EGDMA_{0.8}) was conducted. The emulsions were loaded with Amphotericin B, an antifungal drug used to treat fungal keratitis, and Cyclosporin A, an immunosuppressant used to treat keratoconjunctivitis (Dry Eye Syndrome). The drugs were loaded at topical dose concentrations, Amphotericin B at 0.15% w/v and Cyclosporin A at 0.05% w/v.

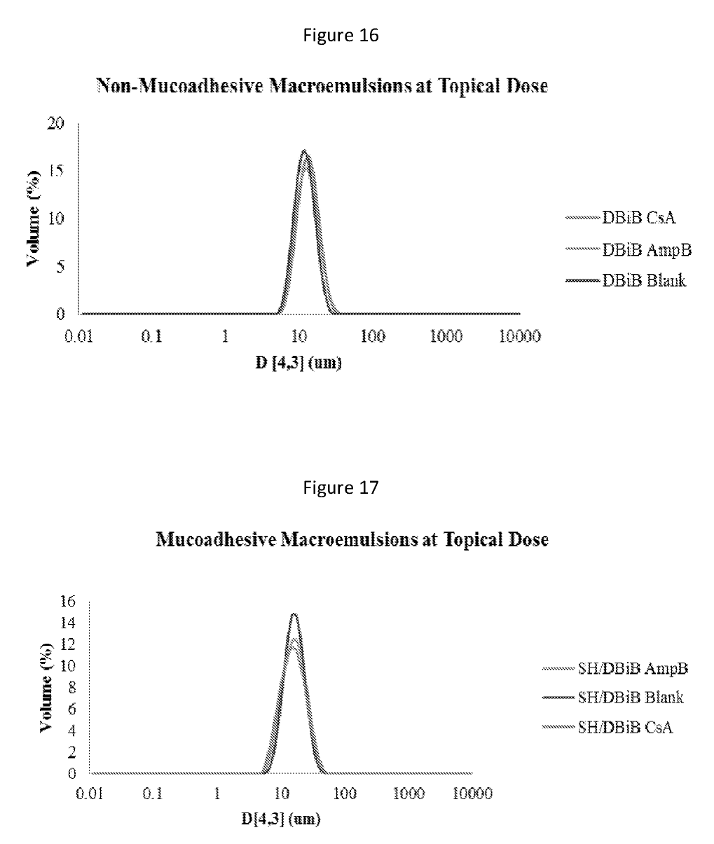

FIG. 16 shows the particle size distributions of the above-mentioned non-mucoadhesive macroemulsions (a blank emulsion, one loaded with Amphotericin B, and one loaded with Cyclosporin A).

FIG. 17 shows the particle size distribution of the above-mentioned mucoadhesive macroemulsions (a blank emulsion, one loaded with Amphotericin B, and one loaded with Cyclosporin A).

The following table shows a comparison of the volume mean diameter of the macroemulsions loaded with Amphotericin B and Cyclosporin A against a blank emulsion. Emulsions stabilised with either DBiB₂₅/SHG₀-BiB₇₅(pOEGMA₅₀-co-EGDMA_{0.8}) (mucoadhesive) or DBiB(pOEGMA₅₀-co-EGDMA_{0.8}) (non-mucoadhesive)

| Polymer | Loading | Diameter (μm) |
|---|---|---|
| DBiB₂₅/SHG₀-BiB₇₅(pOEGMA₅₀-co- | Blank | 15 |
| EGDMA_{0.8}⁾ | AmpB | 16 |
| | CsA | 16 |
| DBiB(pOEGMA₅₀-co-EGDMA_{0.8}) | Blank | 12 |
| | AmpB | 13 |
| | CsA | 13 |

Nanoemulsion Formulations

Nanoemulsions were formulated using the solvent evaporation technique as described above. Amphotericin B and Cyclosporin A were loaded in the oil phase at the clinical topical dose concentration, 0.15% w/v and 0.05% w/v respectively.

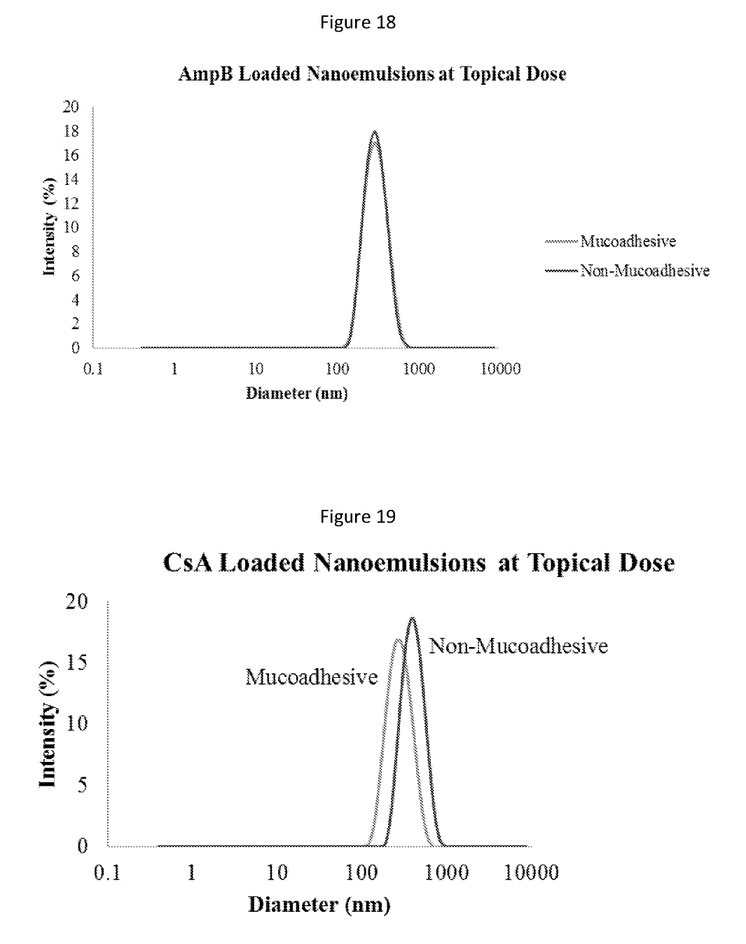

FIG. 18 shows the particle size distributions of the Amphotericin B loaded nanoemulsions (both non- and mucoadhesive), analysed by dynamic light scattering.

FIG. 19 shows the particle size distributions of the Cyclosporin A loaded nanoemulsions (both non- and mucoadhesive), analysed by dynamic light scattering.

The following table shows a comparison of the hydrodynamic diameter and polydispersity (PdI) of nanoemulsions loaded with Amphotericin B and Cyclosporin A against a blank emulsion. Emulsions stabilised with either DBiB₂₅/SHG₀-BiB₇₅(pOEGMA₅₀-co-EGDMA_{0.8}) (mucoadhesive) or DBiB(pOEGMA₅₀-co-EGDMA_{0.8}) (non-mucoadhesive)

| Polymer | Loading | Diameter (nm) | PdI |
|---|---|---|---|
| DBiBₓ/SHG₀- | Blank | 249 | 0.10 |
| BiB_{y}(pOEGMA₅₀-co- | AmpB | 290 | 0.15 |
| EGDMA_{0.8}⁾ | CsA | 264 | 0.07 |
| DBiB(pOEGMA₅₀-co-- | Blank | 250 | 0.06 |
| EGDMA_{0.8}) | AmpB | 290 | 0.17 |
| | CsA | 371 | 0.08 |

Fungal Kill

Agar petri dishes were coated with candida albicans and left to incubate at 35° C. A drop of amphotericin B loaded nanoemulsion was placed onto the petri dish at three concentrations and assessed against two controls, blank emulsion and fungizone, a current manufactured amphotericin B product. The experiment was repeated in triplicate and incubated at 35° C.

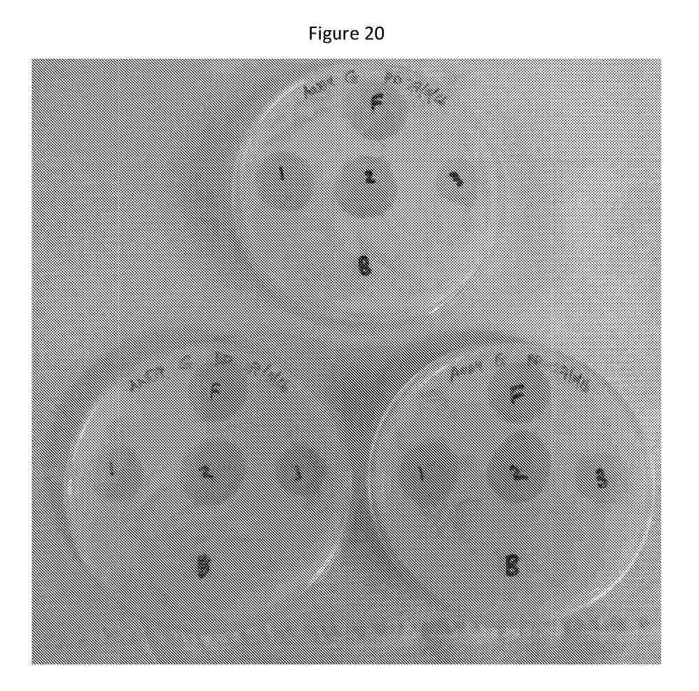

FIG. 20 shows an Amphotericin B fungus kill study with candida albicans, assessing the kill level of the emulsions at varying concentrations. From top of petri dish then left to right; F) Fungizone negative control, 1) 194 mg/mL, 2) 282 mg/mL, 3) 4117 mg/mL and B) blank emulsion positive control.

Cytotoxicity Studies

Overall cytotoxicity of both muco- and non-muocadhesive nanoemulsions was determined from resazurin assays. The Human Corneal Epithelial cells-transformed (HCE-t) were exposed to blank nanoemulsions over a range of dilutions in DMEM:F12 media with 10% FCS. Confluent cells were treated for 1 hr, 4 hrs and 24 hrs to represent maximum dosing time of Amphotericin B and Cyclosporin A systems to the eye.

Controls include: Negative control—healthy cells treated solely with media, Positive control—cells treated with 100% DMSO to induce cell death from 1 hr onwards, and cells only to assure there is no background noise on the fluorescent plate reader.

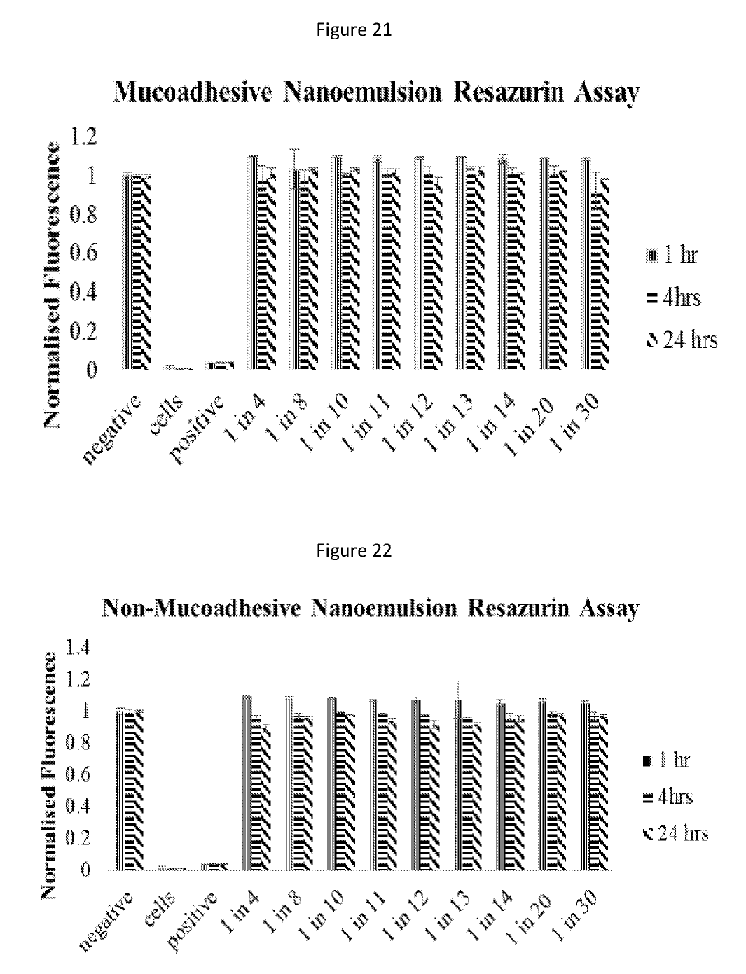

FIG. 21 shows overall cytotoxicity of mucoadhesive nanoemulsion determined from reszaurin assay. HCE-t cells exposed over a range of dilutions of emulsion in media.

FIG. 22 shows overall cytotoxicity of non-mucoadhesive nanoemulsion determined from reszaurin assay. HCE-t cells exposed over a range of dilutions of emulsion in media.

Furthermore, phalloidin staining images of cells treated with Cyclosporin A—loaded emulsions showed non-cytotoxic results:

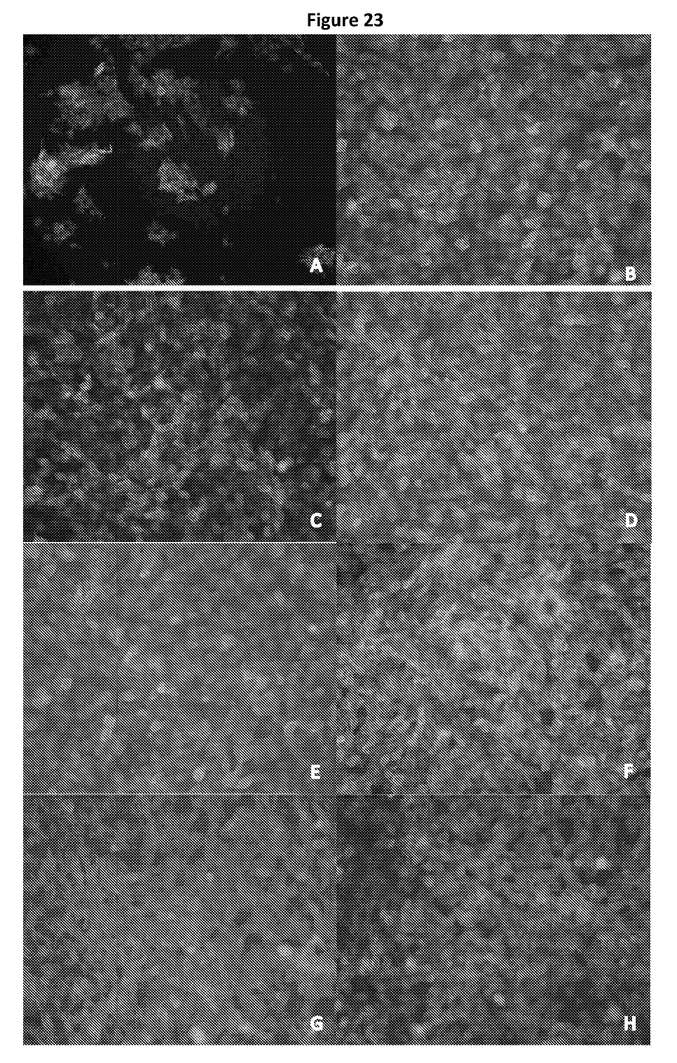

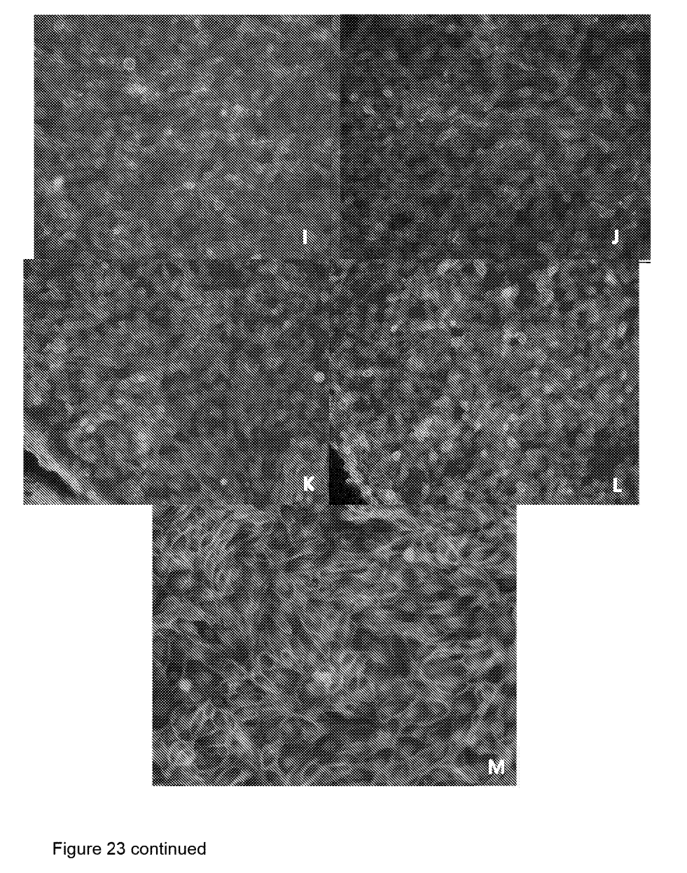

FIG. 23 shows Human Corneal Epithelium Cells transformed stained with phalloidin (green) and 4′, 6-diamidino-2-phenylindole (DAPI). From top, left to right—A) Concentrated emulsion, B) 1 in 2 dilution, C) 1 in 4, D) 1 in 6, E) 1 in 8, F) 1 in 10, G) 1 in 11, H) 1 in 12, I) 1 in 13, J) 1 in 14, K) 1 in 20, L) 1 in 30, M) media only. Images at 20× magnification, Nikon TI-E microscope. Human corneal epithelium cells (HCE-t) were seeded onto a 48 well plate at a density of 15,000 cells/well, and left to establish a monolayer for four days. Nanoemulsion loaded with Cyclosporin A at the topical dose (0.05% w/v) was applied to the cells in triplicate over a range of serial dilutions of emulsion in media (DMEM:F12, 10% FCS), and incubated at 37° C. for 24 hours. After incubation, the emulsion was removed and the cells washed with phosphate buffered saline, PBS, (500 μL) then fixed with neutral buffered formalin (NBF, 10% formalin, approx. 4% formaldehyde), for 10 minutes. NBF was removed and the cells washed again with PBS. The cytotoxicity of the cyclosporin A loaded emulsion was then determined by staining of the cells with phalloidin, to assess the structure of the cell cytoskeleton by fluorescence microscopy. DAPI is a blue fluorescent DNA stain, which binds to the adenine-thymine rich areas, so is able to stain the nuclei of the cells.

1. A branched amphiphilic polymer, suitable for stabilizing an emulsion, comprising:
a. a plurality of polymer chains comprising hydrophobic chain ends;
b. a plurality of polymer chains comprising functional chain ends capable of associating to a biological substrate; and
c. a plurality of branching units.

2. The branched amphiphilic polymer according to claim 1, wherein the functional chain ends form a strong association to mucous or a mucous membrane.

3. The branched amphiphilic polymer according to claim 1, wherein the polymer chains are made from vinyl monomers.

4. The branched amphiphilic polymer according to claim 3, wherein the polymer chains comprise one or more monomers selected from oligo(ethylene glycol) methacrylate (OEGMA), N-butyl methacrylate, N-butyl acrylate, N-butyl methacrylamide 2-hydroxypropyl methacrylate, 2-hydroxypropyl acrylate, N,N-diethyl amino ethyl methacrylate, N,N-diethyl amino ethyl acrylate, glycerol methacrylate, glycerol acrylate and 2-methacryloyloxyethyl phosphorylcholine.

5. The branched amphiphilic polymer according to claim 1, wherein the hydrophobic chain ends of the polymer chains are alkyl chains of 5 carbon atoms or more.

6. The branched amphiphilic polymer according to claim 5, wherein said alkyl chains are initiator residues wherein the initiator is selected from dodecyl bromo isobutyrate (DBiB), hexyl bromo isobutyrate (HBiB) and ethyl bromo isobutyrate (EBiB).

7. The branched amphiphilic polymer according to claim 1, wherein the functional chain ends of the polymer chains comprise one or more thiol groups.

8. The branched amphiphilic polymer according to claim 7, wherein the thiol groups are incorporated into the chain ends of the polymer by way of xanthate groups.

9. A branched amphiphilic polymer according to claim 1, wherein the branching units comprise EGDMA monomers.

10. The branched amphiphilic polymer according to claim 1, wherein the polymer chains comprising hydrophobic chain ends are DBiB(pOEGMA₅₀), said polymer chains comprising functional chain ends are Xan₁-G₀-BiB(pOEGMA₅₀) and the branching units are EGDMA monomers.

11. The branched amphiphilic polymer according to claim 1, wherein at least 50%, 60% or 70% of the polymer chain ends carry functional chain ends.

12. The branched amphiphilic polymer according to claim 1, wherein about 75% of the polymer chain ends carry functional chain ends.

13. A pharmaceutical composition comprising the branched amphiphilic polymer of claim 1, an effective amount of a biologically active agent or a pharmaceutically acceptable salt thereof, and at least one pharmaceutically acceptable carrier, diluent or excipient.

14. The pharmaceutical composition according to claim 13, wherein the composition is an emulsion formulation.

15. The pharmaceutical composition according to claim 14, wherein the composition is an oil-in-water emulsion and the droplet size of the oil-in water emulsion formulation as measured by the z-average diameter and determined by dynamic light scattering is less than 1000 nm or between 1-100 μm.

16. The pharmaceutical composition according to claim 15, wherein the oil-in water emulsion formulation is stable but capable of breaking down to release its contents upon contact and association with a mucous, a mucous membrane, or other biological substrate.

17. The pharmaceutical composition according to claim 16, wherein the composition is an oil-in-water emulsion and the droplet size of the oil-in water emulsion formulation as measured by the z-average diameter and determined by dynamic light scattering is between 1-100 μm.

18. (canceled)

19. A method of preparing an oil-in-water emulsion pharmaceutical composition, comprising mixing an oil phase optionally containing a hydrophobic biologically active agent or a pharmaceutically acceptable salt thereof, or other hydrophobic compound, with an aqueous phase in the presence of an emulsifier, wherein said emulsifier is a branched amphiphilic polymer according to claim 1.

20. A method of treatment comprising the administration of a pharmaceutical composition as claimed in claim 13 to a patient in need thereof.
